(19) United States
(12) Patent Application Publication
(10) Pub. No.: US 2005/0090527 A1
(43) Pub. Date: Apr. 28, 2005

(54) Combination of H1, H3 and H4 receptor antagonists for treatment of allergic and non-allergic pulmonary inflammation, congestion and allergic rhinitis

(75) Inventors: John C. Anthes, Cranford, NJ (US); Robert E. West, Cranford, NJ (US); John A. Hey, Randolph, NJ (US); Robert G. Aslanian, Rockaway, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/764,780

(22) Filed: Jan. 26, 2004

Related U.S. Application Data
(60) Provisional application No. 60/443,207, filed on Jan. 28, 2003.

(51) Int. Cl. A61K 31/4545

(52) U.S. Cl. 514/316

(57) ABSTRACT

The present invention includes methods for treating allergic conditions involving the airway by administering histamine receptor antagonists.

This application claims the benefit of U.S. Provisional Patent Application No. 60/443,207, filed Jan. 28, 2003, which is herein incorporated by reference in its entirety.

### FIELD OF THE INVENTION

The present invention provides methods for treating allergic, non-allergic pulmonary and nasal obstructive disease conditions by administration of histamine receptor antagonist combinations.

### BACKGROUND OF THE INVENTION

Allergic rhinitis, pulmonary inflammation and congestion are medical conditions which may be associated with other medical conditions including asthma, chronic obstructive pulmonary disease (COPD), seasonal allergic rhinitis and perennial allergic rhinitis. In general, these conditions are mediated, at least in part, by inflammation which may be controlled by antagonizing histamine receptors.

Allergic rhinitis, sometime referred to as “hay fever”, is a common illness affecting an estimated 20-40 million Americans, and resulting in 10 million lost days of school or work each year. Two types of allergic rhinitis include seasonal allergic rhinitis and perennial allergic rhinitis. Similarly, congestion, particularly sinus congestion is characterized by inflammation of the tissues in the sinus cavities. Common remedies for rhinitis are “antihistamine” H1 receptor antagonists such as chlorpheniramine maleate.

COPD, asthma and repeated episodes of pulmonary inflammation can lead to alveolar damage and fibrosis which can lead to impaired lung capacity and gas exchange. In general, exposure of the lungs to allergens may lead to mast cell mediated release of histamine and other substances which, in turn, begins a cascade of events leading to inflammation.

U.S. Pat. Nos. 5,217,986 and 5,352,707 to Pomponi et al. attribute an ability for treating conditions including rhinitis and airway congestion to certain compounds apparently having H3 receptor binding activity, but no H1 receptor antagonist activity. No clinical observation or other support is provided for this proposition.

International Patent Application Publication No. WO 02/56871 discloses, generally, the use of a combination of histamine H1 and H4 receptor antagonists for treating allergic disorders and diseases. The publication does not, however, exemplify any particular H1 or H4 receptor antagonists which may be useful for this purpose.

International Patent Application Publication No. WO 98/06394 discloses the use of a combination of histamine H1 and H3 receptor antagonists for treating upper airway allergic responses. The Application does not disclose use of a histamine H4 receptor antagonist.

Further, U.S. Pat. No. 5,869,479 discloses compositions for the treatment of the symptoms of allergic rhinitis using a combination of at least one histamine H1 receptor antagonist and at least one histamine H3 receptor antagonist. The patent does not mention use of an histamine H4 receptor antagonist.

Accordingly, there is a need in the art for an effective method by which to treat or prevent medical conditions such as allergic rhinitis, pulmonary inflammation and congestion by antagonizing histamine receptors such as H1, H3 and H4.

### SUMMARY OF THE INVENTION

The present invention provides a method for treating or preventing an allergic or non-allergic condition characterized by airway inflammation (e.g., allergic rhinitis, congestion or pulmonary inflammation) in a subject (e.g., a human) comprising administering one or more histamine H3 receptor antagonists, one or more histamine H4 receptor antagonists and, optionally, one or more histamine H1 receptor antagonists to the subject. One or more of the antagonists may be combined with a pharmaceutically acceptable carrier in a pharmaceutical composition (e.g., pill, tablet, capsule). Furthermore, substances which antagonize multiple histamine receptors may be used in the present invention. For example, a subject can be administered one or more dual H3H4 antagonists and, optionally, one or more H1 antagonists.

Also provided are combinations comprising one or more substances which antagonize the histamine H3 receptor, in association with one or more substances which antagonize histamine H4 receptor and, optionally, in association with one or more substances which antagonize histamine H1 receptor as well as pharmaceutical compositions, which comprise a pharmaceutically acceptable carrier, thereof. Pharmaceutical compositions are preferably in the form of a pill, capsule or tablet. Preferred combinations comprise one or more H3 receptor antagonists, in association with one or more H4 receptor antagonists, or, alternatively, one or more dual H3/H4 receptor antagonists, in association with one or more H1 receptor antagonists. Another preferred combination comprises one or more dual H1/H3 antagonists in association with one or more H4 antagonists. Preferred antagonists are discussed, in detail, infra.

Preferably, one or more histamine H3 receptor antagonists are selected from thioperamide, impromidine, burimamide, clobenpropit, impentamine, mifetidine, clozapine, S-sopromidine, R-sopromidine, ciproxifam, SKF-91486 (3-(imidazole-4-yl)-propylguanidine sulfate), GR-175737 (Clitherow, et al., (1996) Bioorg. Med. 6: 833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50), ABT331440 (PCT Publication No. WO 02/06223),

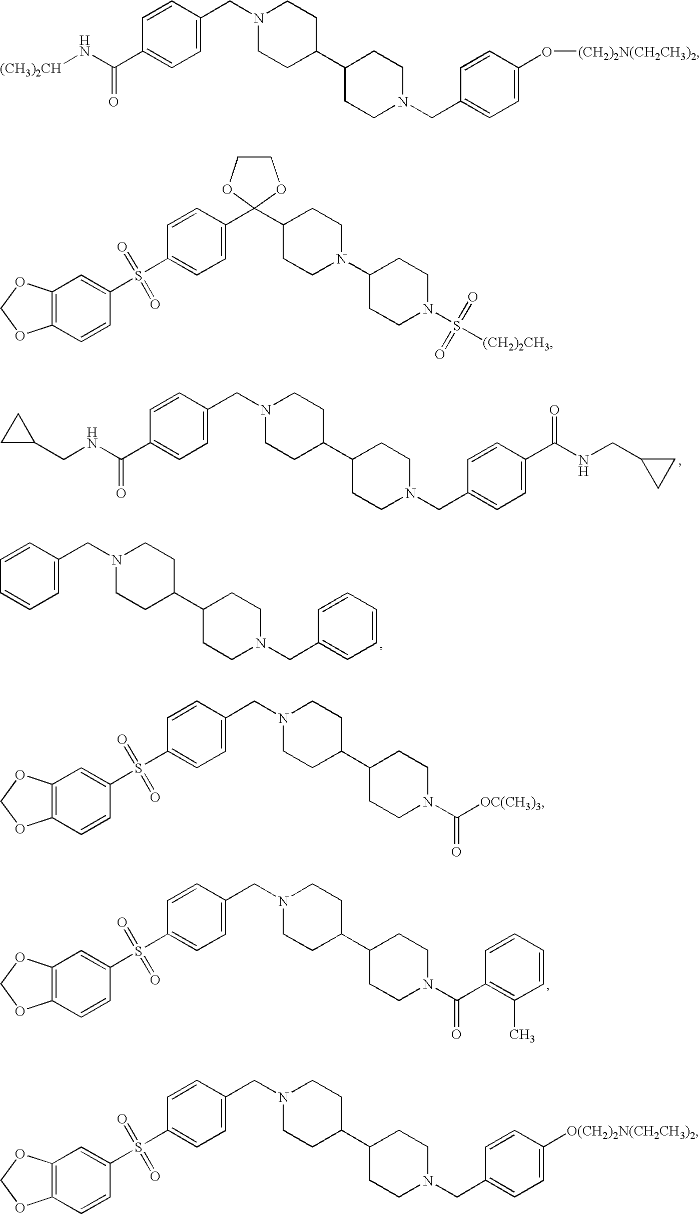

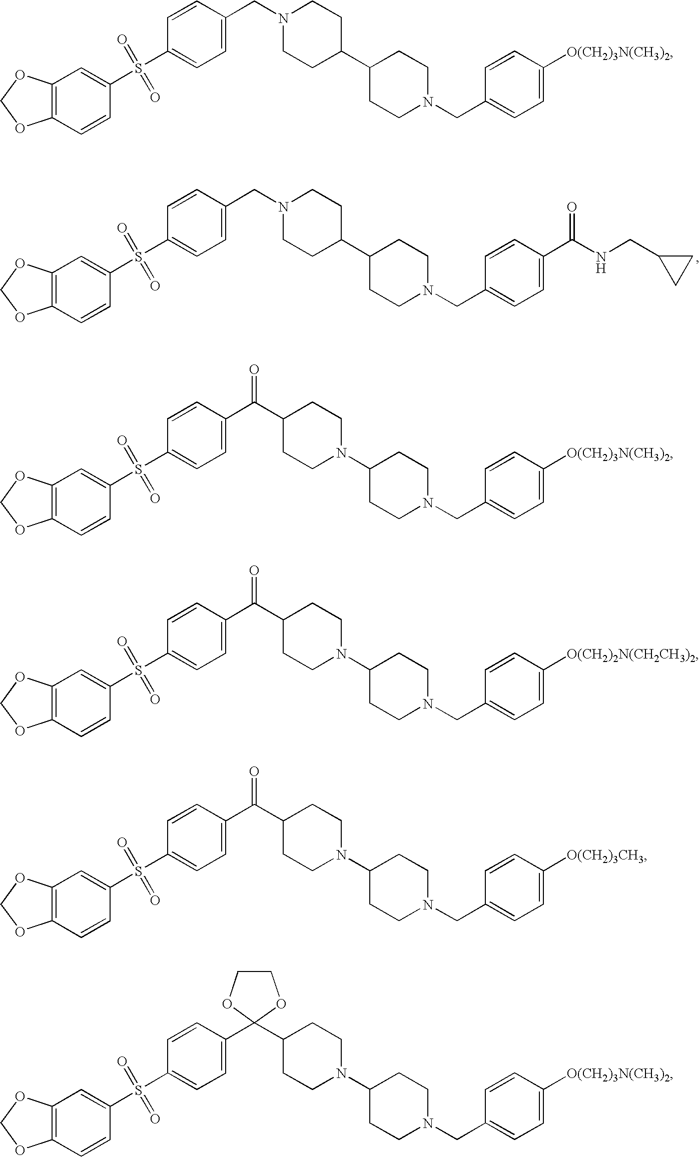

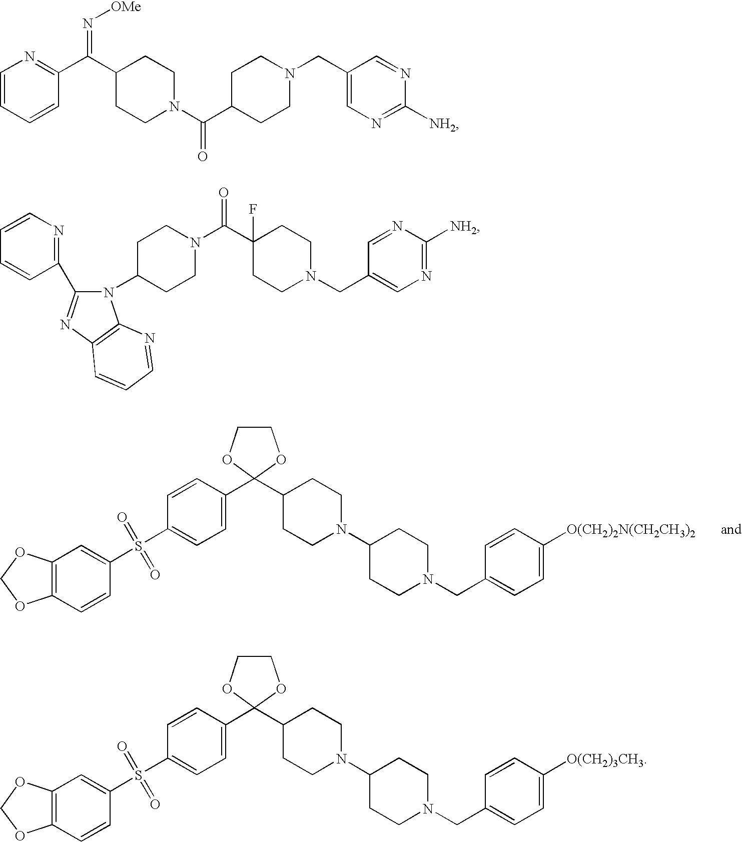

Preferably, one or more dual histamine H3 receptor/histamine H4 receptor antagonists are selected from

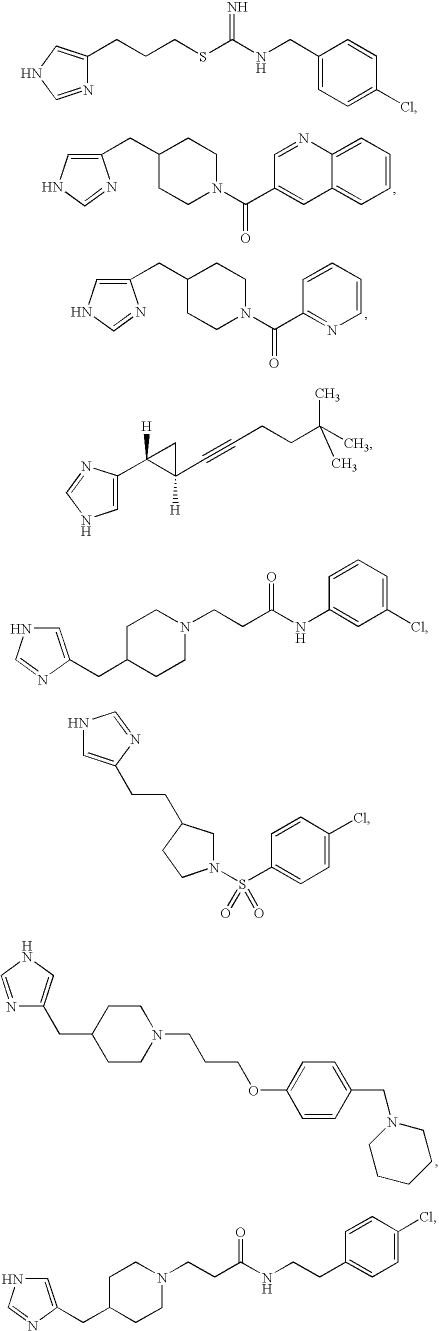

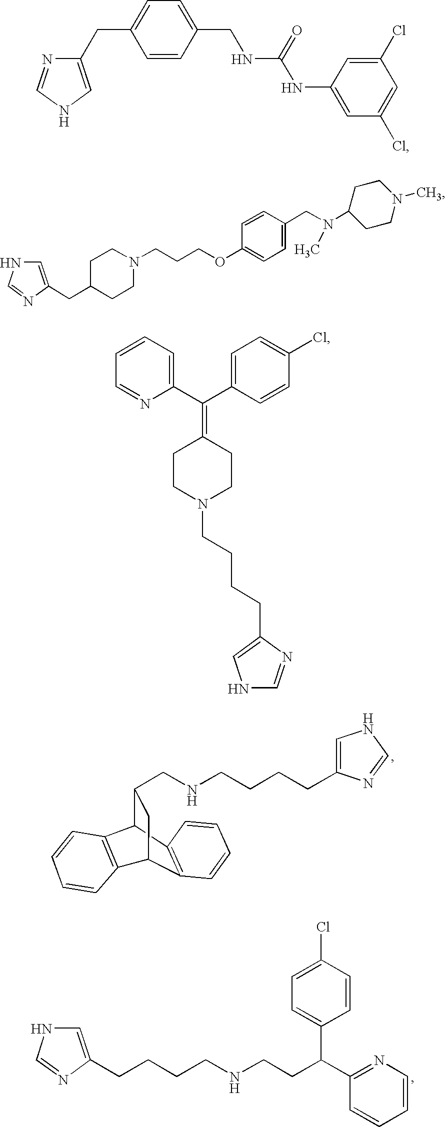

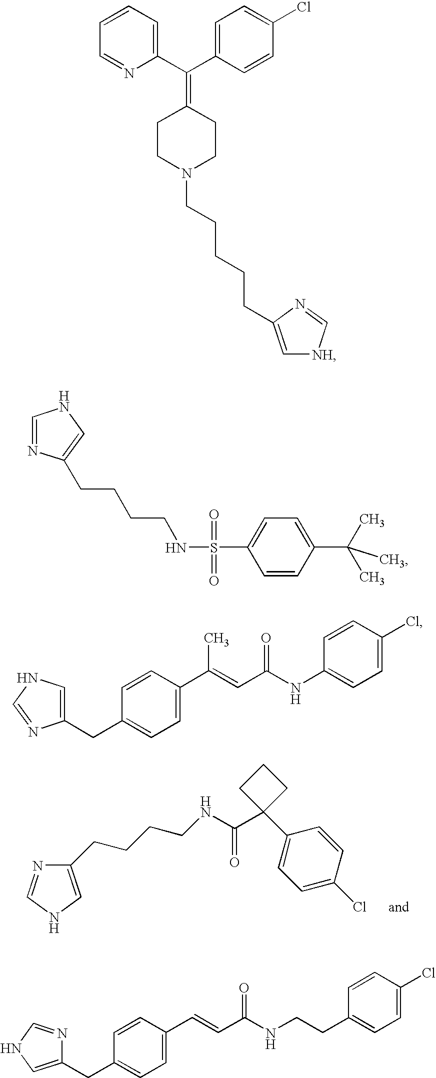

Preferably, one or more histamine H1 receptor antagonists are selected from astemizole, azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norasternizole, picumast, pyrilamine, promethazine, terfenadine, tripelennamine, temelastine, trimeprazine, triprolidine and

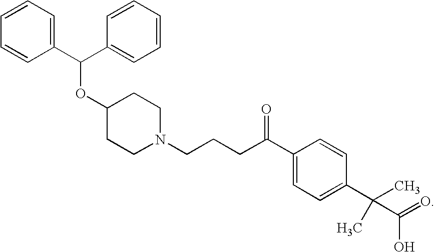

More preferably, one or more histamine H1 receptor antagonists are selected from loratadine, desloratadine, cetirizine and fexofenadine.

The antagonists of the present invention may be administered to the subject by any mode, such as parenterally or non-parenterally. Furthermore, the antagonists may be administered in a single composition.

### DETAILED DESCRIPTION OF THE INVENTION

The present invention includes methods for administering one or more histamine receptor antagonists for the treatment or prevention of diseases and conditions which are mediated by the histamine receptors (e.g., allergic rhinitis, congestion and pulmonary inflammation, preferably associated with asthma, chronic obstructive pulmonary disease (COPD), seasonal allergic rhinitis and perennial allergic rhinitis). Any antagonist or combination of antagonists which antagonize the histamine H3 receptor, the histamine H4 receptor and, optionally, the histamine H1 receptor may be administered to a subject for the purposes of the present invention. The antagonists can antagonize one or more histamine receptors. For example, a subject can be administered a single substance which antagonizes both a histamine H3 receptor and a histamine H4 receptor (dual H3H4 antagonist) and optionally, an additional histamine H1 receptor antagonist.

The terms “H1” and “H1 receptor” both refer to a histamine H1 receptor. The terms “H3” and “H3 receptor” both refer to a histamine H3 receptor. The terms “H4” and “H4 receptor” both refer to a histamine H4 receptor.

The histamine H1 receptors, histamine H3 receptors and histamine H4 receptors of the invention may be from any organism, preferably a mammal (e.g., horse, dog, cat, rat, mouse, rabbit, horse, pig and guinea pig) and most preferably a human. Genbank Accession No. AY136743 discloses a typical human histamine receptor H1, Genbank Accession No. AB045369 discloses a typical human histamine receptor H3 and Genbank Accession No. NM021624 discloses a typical human histamine receptor H4. Moreover, U.S. Pat. No. 6,204,017 discloses a human histamine H4 receptor in SEQ ID NOs: 1 and 2 (SP9144).

The term “subject” includes any organism, preferably a mammal (e.g., horse, dog, cat, rat, mouse, rabbit, horse, pig and guinea pig) and most preferably a human.

The term “in association with” indicates that the components of the combinations of the invention can be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously at several intervals over a given period of time. Moreover, the separate components may be administered to a subject by the same or by a different route (e.g., orally, intranasally, intravenously).

### Histamine Receptor Antagonists

Histamine H3 receptor antagonists include, without limitation: thioperamide, impromidine, burimamide, clobenpropit, impentamine, mifetidine, clozapine, S-sopromidine, R-sopromidine, ciproxifam, SKF-91486 (3-(imidazole-4-yl)-propylguanidine sulfate), GR-175737 (Clitherow, et al., (1996) Bioorg. Med. 6: 833-838), GT-2016 (Tedford, et al., (1995) J. Pharm. Exp. Ther 275(2): 596-604), GT-2331 (Tedford, et al., (1998) Eur. J. Pharmacol. 351(3): 307-11), GT-2394 (Yates, et al., (2000) Soc. Neurosci. Abstr. 26: 279.), JB98064 (Linney, et al., (2000) J. Med. Chem. 43: 2362-2370), UCL-1199 (Ganellin, et al., (1995) J. Med. Chem. 38(17): 3342-50) and ABT331440 (PCT Publication No. WO 02/06223;

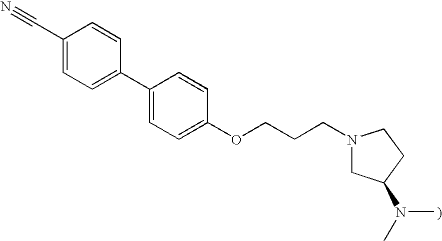

Other exemplary histamine H3 receptor antagonists are set forth, below, in Table 1.

TABLE 1
Histamine H3 Receptor Antagonists.

| Formula | Structure |
|---|---|
| 1 | 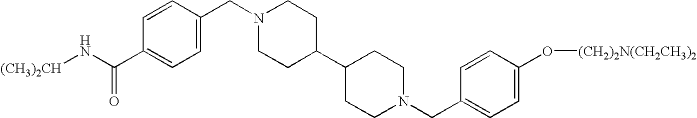 |
| 2 | 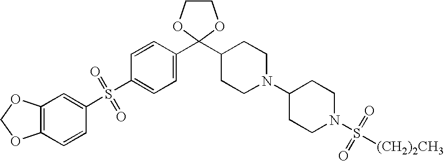 |
| 3 | 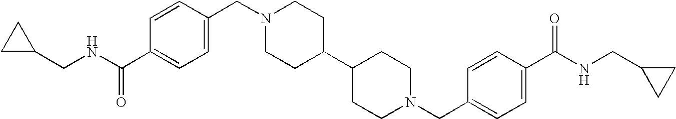 |
| 4 | 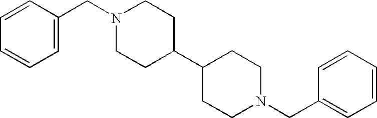 |
| 5 | 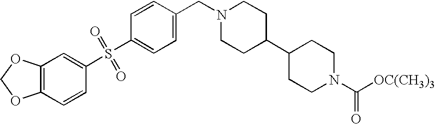 |
| 6 | 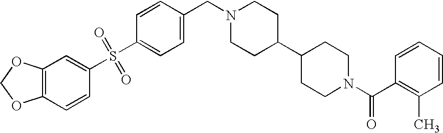 |
| 7 | 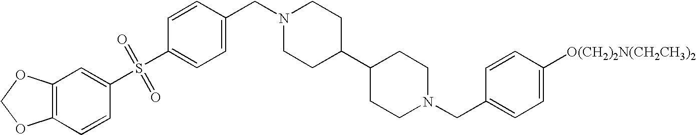 |
| 8 | 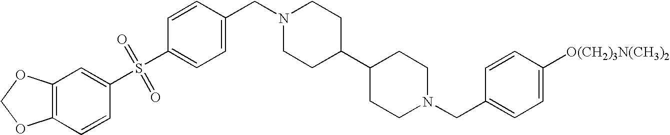 |
| 9 | 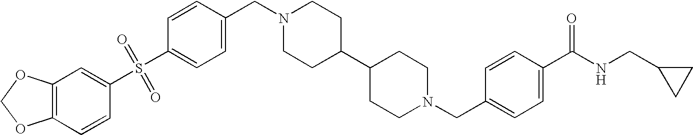 |
| 10 | 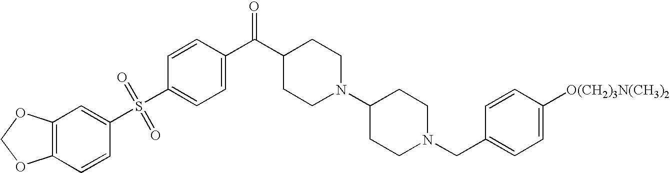 |
| 11 | 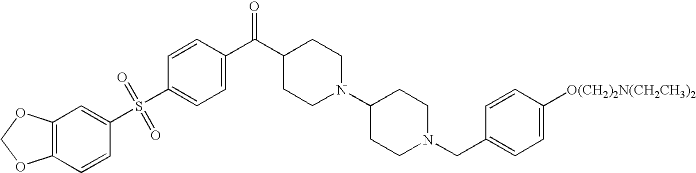 |
| 12 | 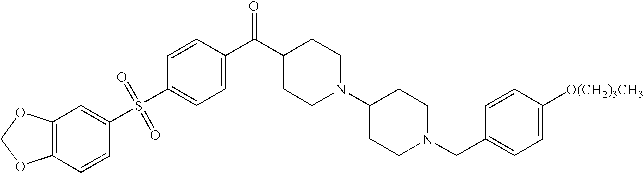 |
| 13 | 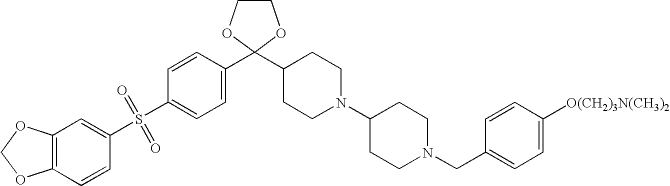 |
| 14 | 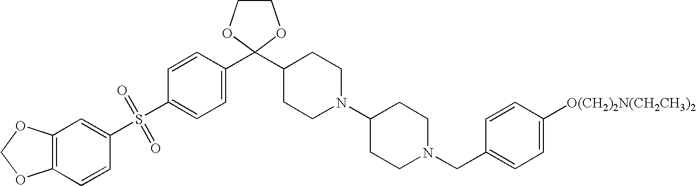 |
| 15 | 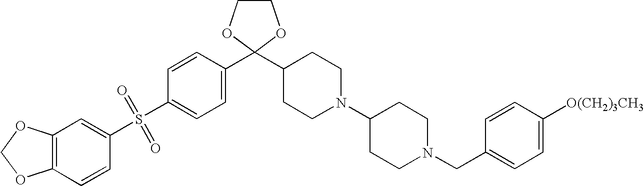 |
| 16 | 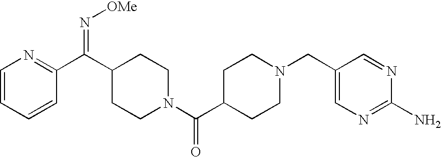 |
| 17 | 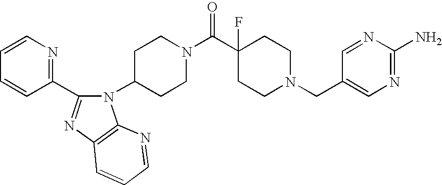 |

Exemplary, dual H3/H4 receptor antagonists are shown, below, in Table 2.

TABLE 2
Dual H3/H4 Receptor Antagonists.

| Formula | Structure |
|---|---|
| 18 | 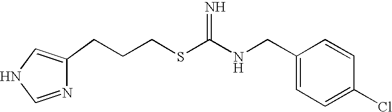 |
| 19 | 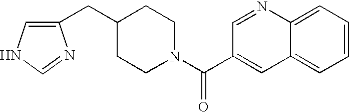 |
| 20 | 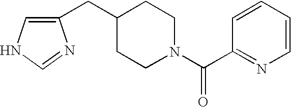 |
| 21 | 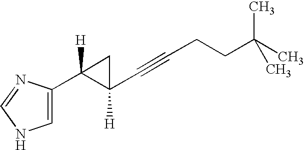 |
| 22 | 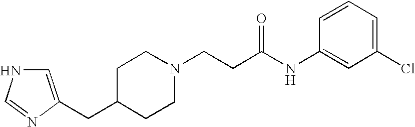 |
| 23 | 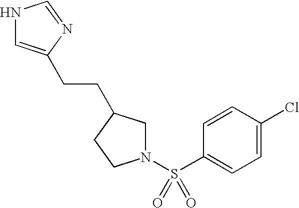 |
| 24 | 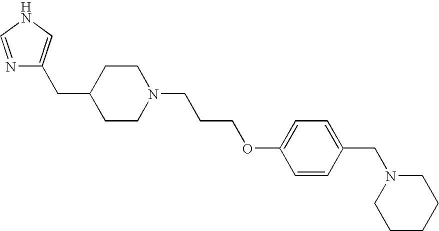 |
| 25 | 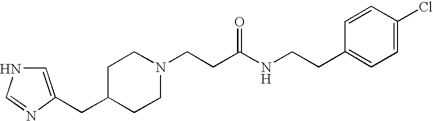 |
| 26 | 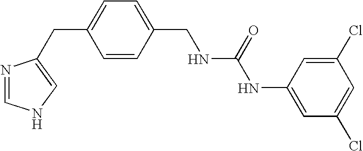 |
| 27 | 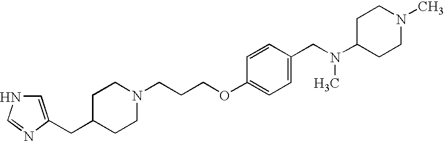 |
| 28 | 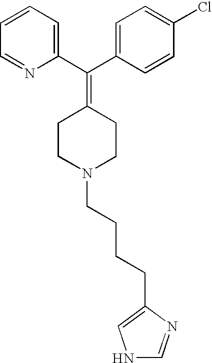 |
| 29 | 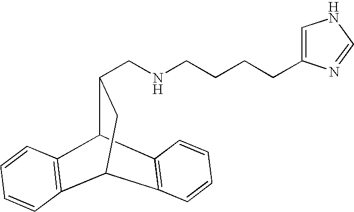 |
| 30 | 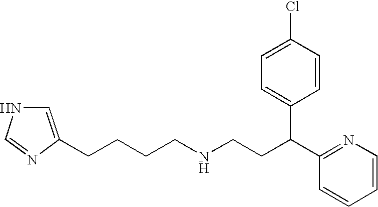 |
| 31 | 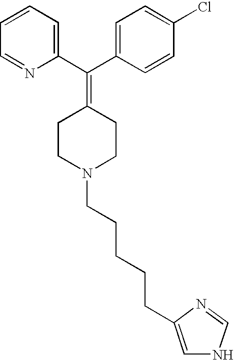 |
| 32 | 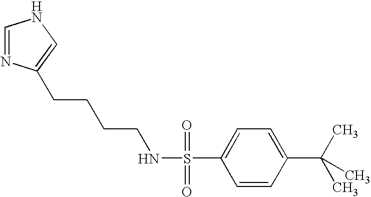 |
| 33 | 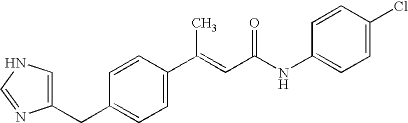 |
| 34 | 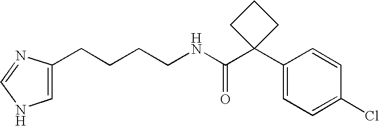 |
| 35 | 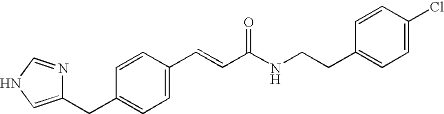 |

Preferably, the dual H3/H4 receptor antagonist is selected from compounds comprising a formula selected from formulas 18, 19, 20, 20, 22, 23, 24, 26, 28, 31, 32, 33 and 35.

A H4 receptor antagonist can also be any one or more of those disclosed in Jablonowski et al., J. Med. Chem. 46:3957-3960 (2003), particularly compound 6, and/or compound 10e and/or compound 101 therein.

Numerous chemical substances are known to have histamine H1 receptor antagonist activity. Many useful compounds can be classified as ethanolamines, ethylenediamines, alkylamines, phenothiazines or piperidines. Representative H1 receptor antagonists include, without limitation: astemizole, cetirizine, azatadine, azelastine, acrivastine, brompheniramine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norasternizole, picumast, pyrilamine, promethazine, terfenadine, tripelennamine, temelastine, trimeprazine, triprolidine and the compound of formula 36:

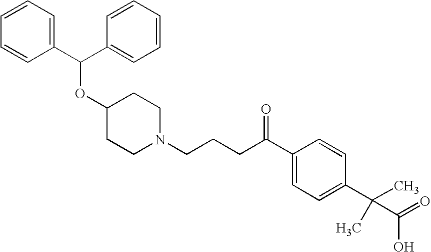

Histamine H3 receptor antagonists which are part of the present invention are disclosed in several U.S. patents, applications and publications:

PCT Publication No. WO 02/72570 discloses compounds comprising the following structural formula: 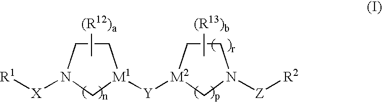
or a pharmaceutically acceptable salt or solvate thereof, wherein:
   (A) R¹ is selected from:
      (1) aryl;
      (2) heteroaryl;
      (3) heterocycloalkyl
      (4) alkyl;
      (5) —C(O)N(R^{4B})₂;
      (6) cycloalkyl;
      (7) arylalkyl;
      (8) heteroarylheteroaryl (e.g., isoxazoylthienyl or pyridylthienyl); or
      (9) a group selected from: 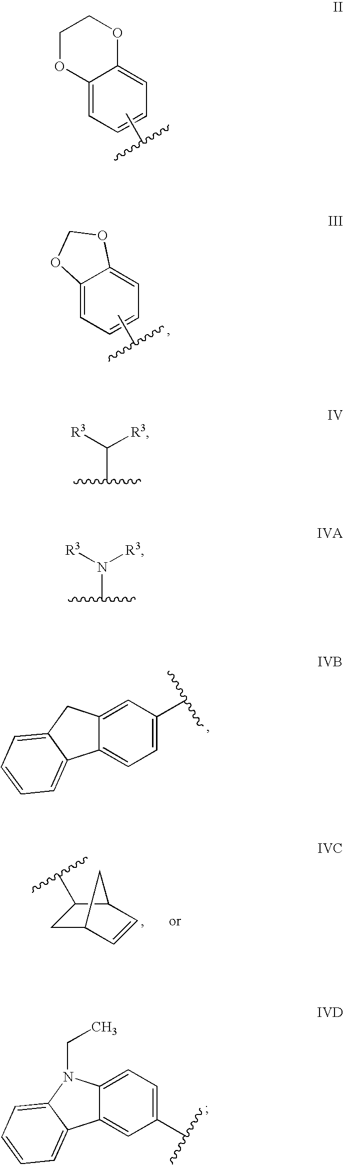
said aryl (see (A)(1) above), heteroaryl (see (A)(2) above), aryl portion of arylalkyl (see (A)(7) above), phenyl ring of formula It (see (A)(9) above), phenyl ring of formula III (see (A)(9) above), phenyl rings of formula IVB (see (A)(9) above), or phenyl rings of formula IVD (see (A)(9) above) are optionally substituted with 1 to 3 substituents independently selected from:
   (1) halogen (e.g. Br, F, or Cl, preferably F or Cl);
   (2) hydroxyl (i.e., —OH);
   (3) lower alkoxy (e.g., C₁ to C₆ alkoxy, preferably C₁ to C₄ alkoxy, more preferably C₁ to C₂ alkoxy, most preferably methoxy);
   (4) —Oaryl (i.e., aryloxy);
   (5) —SR²²;
   (6) —CF₃;
   (7) —OCF₃;
   (8) —OCHF₂;
   (9) —NR⁴R⁵;
   (10) phenyl;
   (11) NO₂,
   (12) —CO₂R⁴;
   (13) —CON(R⁴)₂ wherein each R⁴ is the same or different;
   (14) —S(O)₂R²²:
   (15) —S(O)₂N(R²⁰), wherein each R²⁰ is the same or different;
   (16) —N(R²⁴)S(O)₂R²²;
   (17) —CN;
   (18) —CH₂OH;
   (19) —OCH₂CH₂OR²²;
   (20) alkyl (e.g., C₁ to C₄, such as methyl);
   (21) substituted phenyl wherein said phenyl has 1 to 3 substituents independently selected from alkyl, halogen, —CN, —NO₂, —OCHF₂, —Oalkyl;
   (22) —Oalkylaryl (preferably —Oalkylphenyl or —Oalkyl-substituted phenyl, e.g. —OCH₂dichlorophenyl, such as —OCH₂-2,6-dichlorophenyl or —OCH₂-2-chloro-6-fluorophenyl) wherein said aryl group is optionally substituted with 1 to 3 independently selected halogens; or
   (23) phenyl;
(B) X is selected from alkyl (e.g., —(CH₂)_{q}— or branched alkyl) or —S(O)₂—:
(C) Y represents
   (1) a single bond (i.e., Y represents a direct bond from M¹ to M²); or
   (2) Y is selected from —C(O)—, —C(S)—, —(CH₂)_{q}—, or —NR⁴C(O)—; with the provisos that:
      (a) when M¹ is N, then Y is not —NR⁴C(O)—; and
      (b) when Y is a bond, then M¹ and M² are both carbon;
(D) M¹ and M² are independently selected from C or N;
(E) Z is selected from: C₁-C₆ alkyl, —SO₂—, —C(O)— or —C(O)NR⁴—;
(F) R² is selected from:
   (1) a six-membered heteroaryl ring having 1 or 2 heteroatoms independently selected from N or N—O (i.e., N-oxide), with the remaining ring atoms being carbon;
   (2) a five-membered heteroaryl ring having 1 to 3 heteroatoms selected from nitrogen, oxygen, or sulfur with the remaining ring atoms being carbon; or
   (3) an alkyl group, preferably a C₁ to C₄ alkyl group, more preferably methyl;
   (4) an aryl group, e.g., phenyl or substituted phenyl (preferably phenyl), wherein said substituted phenyl is substituted with 1 to 3 substituents independently selected from: halogen, —Oalkyl, —OCF₃, —CF₃, —CN, —NO₂, —NHC(O)CH₃, or —O(CH₂)_{q}N(R^{10A})₂;
   (5) —N(R^{11A})₂ wherein each R^{11A} is independently selected from: H, alkyl (e.g., i-propyl) or aryl (e.g., phenyl), preferably one R^{11A} is H and the other is phenyl or alkyl (e.g., i-propyl);
   (6) a group of the formula: 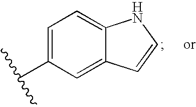
   (7) a heteroarylheteroaryl group, e.g. 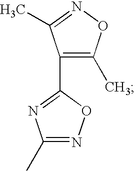
said five membered heteroaryl ring ((F)(2) above) or six-membered heteroaryl ring ((F)(1) above) is optionally substituted with 1 to 3 substituents selected from:
   (a) halogen;
   (b) hydroxyl,
   (c) lower alkyl;
   (d) lower alkoxy;
   (e) —CF₃;
   (f) —NR⁴R⁵;
   (g) phenyl;
   (h) —NO₂;
   (i) —C(O)N(R⁴)₂ (wherein each R⁴ is the same or different);
   (j) —C(O)₂R⁴; or
   (k) phenyl substituted with 1 to 3 substituents independently selected from: halogen, —Oalkyl, —OCF₃, —CF₃, —CN, —NO₂ or —O(CH₂)_{q}N(R^{10A})₂;
(G) R³ is is selected from:
   (1) aryl;
   (2) heteroaryl;
   (3) heterocycloalkyl
   (4) alkyl; or
   (5) cycloalkyl:
wherein said aryl or heteroaryl R³ groups is optionally substituted with 1 to 3 substituents independently selected from:
   (a) halogen (e.g., Br, F, or Cl, preferably F or Cl);
   (b) hydroxyl (i.e., —OH);
   (c) lower alkoxy (e.g., C₁ to C₆ alkoxy, preferably C₁ to C₄ alkoxy, more preferably C₁ to C₂ alkoxy, most preferably methoxy);
   (d) —Oaryl (i.e., aryloxy);
   (e) —SR²²;
   (f) —CF₃;
   (g) —OCF₃;
   (h) —OCHF₂;
   (i) —NR⁴R⁵;
   (j) phenyl;
   (k) —NO₂;
   (l) —CO₂R⁴;
   (m) —CON(R⁴)₂ wherein each R⁴ is the same or different;
   (n) —S(O)₂R²²;
   (o)—S(O)₂N(R²⁰)₂ wherein each R²⁰ is the same or different;
   (p) —N(R²⁴)S(O)₂R²²;
   (q) —CN;
   (r) —CH₂OH;
   (s) —OCH₂CH₂OR²²; or
   (t) alkyl;
(H) R⁴ is selected from:
   (1) hydrogen,
   (2) C₁-C₆ alkyl;
   (3) cycloalkyl;
   (4) cycloalkylalkyl (e.g., cyclopropyl-CH₂— or cyclohexyl-CH₂—);
   (5) heterocycloalkylalky (e.g., tetrahydrofuranyl-CH₂—);
   (6) bridged bicyclic cycloalkyl ring, such as, for example: 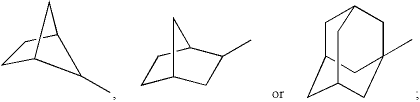
   (7) aryl having a fused heterocycloalkyl ring bound to said aryl ring, preferably the heteroatoms in said heterocycloalkyl ring are two oxygen atoms. e.g., phenyl having a heterocycloalkyl ring bound to said phenyl ring, such as 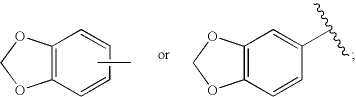
   (8) aryl;
   (9) arylalkyl;
   (10) alkylaryl;
   (11) —(CH₂)_{d}CH(R^{12A})₂ wherein d is 1 to 3 (preferably 1), and each R^{12A} is independently selected from phenyl or substituted phenyl, said substituted phenyl being substituted with 1 to 3 substituents independently selected from: halogen, —Oalkyl, —OCF₃, —CF₃, —CN, or —NO₂, e.g., 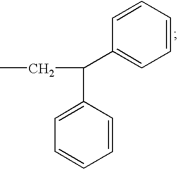
   (12) heterocycloalkylheteroaryl, e.g., 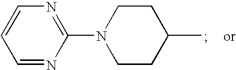
   (13) —(C₁ to C₆)alkylene-O—R²² (e.g., —C₃H₆OCH₃);
      wherein the aryl R⁴ group, the aryl portion of the arylalkyl R⁴ group, or the aryl portion of the alkylaryl R⁴ group is optionally substituted with 1 to 3 substituents independently selected from:
(a) halogen;
(b) hydroxyl;
(c) lower alkyl;
(d) lower alkoxy;
(e) —CF₃:
(f) —N(R²⁰)(R²⁴).
(g) phenyl;
(h) —NO₂;
(i) —C(O)N(R²⁰)₂ (wherein each R²⁰ is the same or different),
(j) —C(O)R²²;
(i) —(CH₂)ₖ-cycloalkyl;
(j) —(CH₂)_{q}-aryl; or
(k) —(CH₂)ₘ—OR²²;
(I) each R^{4B} is independently selected from: H, heteroaryl (e.g., pyridyl), alkyl, alkenyl (e.g., allyl), a group of the formula 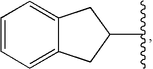
arylalkyl (e.g., benzyl), or arylalkyl wherein the aryl moiety is substituted with 1-3 substituents independently selected from: halogen (e.g. —CH₂-p-Clphenyl); preferably one R^{4B} is H;
(J) R⁵ is selected from: hydrogen, C₁-C₆ alkyl, —C(O)R²⁰ (e.g., —C(O)alkyl, such as —C(O)CH₃), —C(O)₂R²⁰, —C(O)N(R²⁰)₂ (wherein each R²⁰ is the same or different);
(K) each R^{10A} is independently selected from H or C₁ to C₆ alkyl (e.g. methyl), or each R^{10A}, taken together with the nitrogen atom to which they are bound, forms a 4 to 7 membered heterocycloalkyl ring;
(L) R¹² is
   (1) selected from alkyl, hydroxyl, alkoxy, or fluoro, provided that when R¹² is hydroxy or fluoro then R¹² is not bound to a carbon adjacent to a nitrogen; or
   (2) R¹² forms an alkyl bridge from one ring carbon to another ring carbon, an example of such a bridged ring system is: 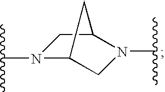
(M) R¹³ is
   (1) selected from alkyl, hydroxyl, alkoxy, or fluoro, provided that when R¹³ is hydroxy or fluoro then R¹³ is not bound to a carbon adjacent to a nitrogen; or
   (2) R¹³ forms an alkyl bridge from one ring carbon to another ring carbon, an example of such a bridged ring system is: 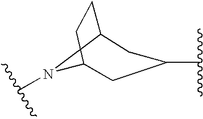
(N)R²⁰ is selected from hydrogen, alkyl, or aryl, wherein said aryl group is optionally substituted with from 1 to 3 groups independently selected from: halogen, —CF₃, —OCF₃, hydroxyl, or methoxy; or when two R²⁰ groups are present, said two R²⁰ groups taken together with the nitrogen to which they are bound form a five or six membered heterocyclic ring;
(O) R²² is selected from: heterocycloalkyl (e.g., morpholinyl or pyrrolidinyl), alkyl or aryl, wherein said aryl group is optionally substituted with 1 to 3 groups independently selected from halogen, CF₃, —OCF₃, hydroxyl, or methoxy;
(P) R²⁴ is selected from: hydrogen, alkyl —SO₂R²², or aryl, wherein said aryl group is optionally substituted with 1 to 3 groups independently selected from halogen, —CF₃, —OCF₃, hydroxyl, or methoxy;
(O) a is 0 to 2;
(R) b is 0 to 2;
(S) k is 1 to 5;
(T) m is 2 to 5;
(U) n is 1, 2 or 3 with the proviso that when M¹ is N, then n is not 1;
(V) p is 1, 2 or 3 with the proviso that when M² is N, then p is not 1;
(W) q is 1 to 5; and
(X) r is 1, 2, or 3 with the proviso that when r is 2 or 3, then M² is C and p is 1.

PCT Publication No WO 02/32893 discloses compounds comprising the following structural formula: 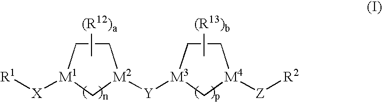
or a pharmaceutically acceptable salt or solvate thereof, wherein:
   (1) R¹ is is selected from:
      (a) aryl;
      (b) heteroaryl;
      (c) heterocycloalkyl
      (d) alkyl;
      (e) cycloalkyl; or
      (f) alkylaryl;
         wherein said R¹ groups are optionally substituted with 1 to 4 substituents independently selected from:
(1) halogen (e.g., Br, F, or Cl, preferably F or Cl);
(2) hydroxyl (i.e., —OH);
(3) lower alkoxy (e.g., C₁ to C₆ alkoxy, preferably C₁ to C₄ alkoxy, most preferably C₁ to C₂ alkoxy, more preferably methoxy);
(4) —CF₃;
(5) CF₃O—;
(6) —NR⁴R⁵;
(7) phenyl;
(8) —NO₂,
(9) —CO₂R⁴;
(10) —CON(R⁴)₂ wherein each R⁴ is the same or different;
(11) —S(O)ₘN(R²⁰)₂ wherein each R₂₀ is the same or different H or alkyl group, preferably C₁ to C₄ alkyl, most preferably C₁-C₂ alkyl, and more preferably methyl;
(12) —CN; or
(13) alkyl; or
(2) R¹ and X taken together form a group selected from: 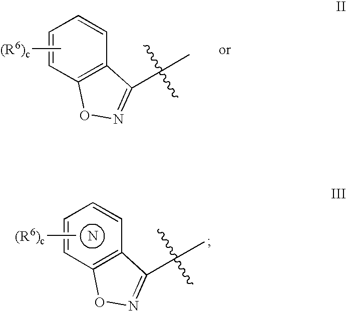
(3) X is selected from: ═C(O), ═C(NOR³), ═C(NNR⁴R⁵), 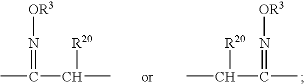
(4) M¹ is carbon;
(5) M² is selected from C or N;
(6) M³ and M⁴ are independently selected from C or N;
(7) Y is selected from: is —CH₂—, ═C(O), ═C(NOR²⁰) (wherein R²⁰ is as defined above), or ═C(S);
(8) Z is a C₁-C₆ alkyl group;
(9) R² is a five or six-membered heteroaryl ring, said six-membered heteroaryl ring comprising 1 or 2 nitrogen atoms with the remaining ring atoms being carbon, and said five-membered heteroaryl ring containing 1 or 2 heteroatoms selected from: nitrogen, oxygen, or sulfur with the remaining ring atoms being carbon; said five or six membered heteroaryl rings being optionally substituted with 1 to 3 substituents independently selected from: halogen, hydroxyl, lower alkyl, lower alkoxy, —CF₃, CF₃O—, —NR⁴R⁵, phenyl, —NO₂, —CO₂R⁴, —CON(R⁴)₂ wherein each R⁴ is the same or different, —CH₂NR⁴R⁵, —(N)C(NR⁴R⁵)₂, or —CN;
(10) R³ is selected from:
   (a) hydrogen;
   (b) C₁-C₆ alkyl;
   (c) aryl;
   (d) heteroaryl;
   (e) heterocycloalkyl;
   (f) arylalkyl (e.g. aryl(C₁ to C₄)alkyl, e.g., —(CH₂)_{w}aryl wherein w is 1 to 4, preferably 1 or 2, and most preferably 1, such as, for example —CH₂phenyl or —CH₂substituted phenyl);
   (g) —(CH₂)ₑ—C(O)N(R⁴)₂ wherein each R⁴ is the same or different,
   (h) —(CH₂)ₑ—C(O)OR⁴;
   (i) —(CH₂)ₑ—C(O)R³⁰ wherein R³⁰ is a heterocycloalkyl group, such as, for example, morpholinyl, piperidinyl, piperazinyl or pyrrolidinyl, including 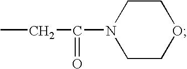
   (j) —CF₃; or
   (k) —CH₂CF₃;
      wherein said aryl, heteroaryl, heterocycloalkyl, and the aryl portion of said arylalkyl are optionally substituted with 1 to 3 (preferably 1) substituents selected from: halogen (e.g., F or Cl), —OH, —OCF₃, —CF₃, —CN, —N(R⁴⁵)₂, —CO₂R⁴⁵, or —C(O)N(R⁴⁵)₂, wherein each R⁴⁵ is independently selected from: H, alkyl, alkylaryl, or alkylaryl wherein said aryl moiety is substituted with 1 to 3 substituents independently selected from —CF₃, —OH, halogen, alkyl, —NO₂, or —CN;
(11) R⁴ is selected from: hydrogen, C₁-C₆ alkyl, aryl, alkylaryl, said aryl and alkylaryl groups being optionally substituted with 1 to 3 substituents selected from: halogen, —CF₃, —OCF₃, —OH, —N(R⁴⁵)₂, —CO₂R⁴⁵, —C(O)N(R⁴⁵)₂, or —CN; wherein R⁴⁵ is as defined above;
(12) R⁵ is selected from: hydrogen, C₁-C₆ alkyl, —C(O)R⁴, —C(O)₂R⁴, or —C(O)N(R⁴)₂ wherein each R⁴ is independently selected, and R⁴ is as defined above;
(13) or R⁴ and R⁵ taken together with the nitrogen atom to which they are bound forms a five or six membered heterocycloalkyl ring (e.g., morpholine);
(14) R⁸ is selected from: alkyl, aryl, alkylaryl, halogen, hydroxyl, lower alkoxy, —CF₃, CF₃O—, —NR⁴R⁵, phenyl, —NO₂, —O₂R⁴, CON(R⁴)₂ wherein each R⁴ is the same or different, or —CN;
(15) R¹² is selected from: alkyl, hydroxyl, alkoxy, or fluoro;
(16) R¹³ is selected from: alkyl, hydroxyl, alkoxy, or fluoro;
(17) a (subscript for R¹²) is 0 to 2;
(18) b (subscript for R¹³) is 0 to 2;
(19) c (subscript for R⁶) is 0 to 2;
(20) e is 0 to 5;
(21) m is 1 or 2;
(22) n is 1, 2 or 3; and
(23) p is 1, 2 or 3, with the proviso that when M³ and M⁴ are both nitrogen, then p is 2 or 3 (i.e., p is not 1 when M³ and M² are both nitrogen).

U.S. Pat. No. 5,463,074 discloses compounds comprising the following structural formula: 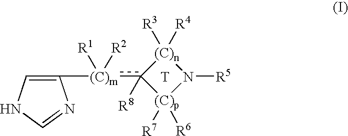
or a pharmaceutically acceptable salt or solvate thereof, wherein:
   (A) m is an integer selected from the group consisting of: 0, 1, and 2;
   (B) n and p are integers and are each independently selected from the group consisting of: 0, 1, 2, and 3 such that the sum of n and p is 2 or 3 such that when the sum of n and p is 2, T is a 4-membered ring and when the sum of n and p is 3, T is a 5-membered ring;
   (C) each R¹, R², R³, R⁴, R⁶, R⁷, and R⁸ is independently selected from the group consisting of:
      (1) H;
      (2) C₁ to C₆ alkyl;
      (3) C₃ to C₆ cycloalkyl; and
      (4) —(CH₂)_{q}—R⁹ wherein q is an integer of: 1 to 7, and R⁹ is selected from the group consisting of: phenyl, substituted phenyl, —OR¹⁰, —C(O)OR₁₀, —C(O)R¹⁰, —OC(O)R¹⁰, —C(O)NR¹⁰R¹¹, CN and —SR¹⁰ wherein R¹⁰ and R¹¹ are as defined below and wherein the substituents on said substituted phenyl are each independently selected from the group consisting of: —OH, —O—(C₁ to C₆)alkyl, halogen, C₁ to C₆ alkyl, —CF₃, —CN, and —NO₂, and wherein said substituted phenyl contains from 1 to 3 substituents; examples of —(CH₂)₁—R⁹ include benzyl, substituted benzyl and the like, wherein the substituents on the substituted benzyl are as defined above for said substituted phenyl;
   (D) R⁵ is selected from the group consisting of:
      (1) H;
      (2) C₁ to C₂₀ alkyl;
      (3) C₃ to C₆ cycloalkyl;
      (4) C(O)OR^{10′}; wherein R^{10′} is the same as R¹⁰ defined below except that R^{10′} is not H;
      (5) —C(O)R¹⁰;
      (6) —C(O)NR¹⁰R¹¹;
      (7) allyl;
      (8) propargyl; and
      (9) —(CH₂)_{q}—R⁹, wherein q and R⁹ are as defined above with the proviso that when q is 1 when R⁹ is not —OH or —SH;
   (E) R¹⁰ and R¹¹ are each independently selected from the group consisting of: H, C₁ to C₆ alkyl, and C₃ to C₆ cycloalkyl; and, for the substituent —C(O)NR¹⁰R¹¹, R¹⁰ and R¹¹, together with the nitrogen to which they are bound, can form a ring having 5, 6, or 7 atoms;
   (F) the dotted line ( . . . ) represents a double bond that is optionally present when m is 1, and T is a 5-membered ring, and n is not 0, and p is not 0 (i.e., the nitrogen in the ring is not bound directly to the carbon atom bearing the double bond), and when said double bond is present then R² and R⁸ are absent;
   (G) when m is 2, each R¹ is the same or different substituent for each m, and each R² is the same or different substituent for each m;
   (H) when n is 2 or 3, each R³ is the same or different substituent for each n, and each R⁴ is the same or different substituent for each n, and
   (I) when p is 2 or 3, each R⁶ is the same or different substituent for each p, and each R⁷ is the same or different substituent for each p.

U.S. Pat. No. 5,633,250 discloses compounds comprising the following structural formula: 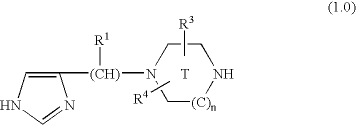
or a pharmaceutically acceptable salt or solvate thereof, wherein:
   (A) n is 1 or 2, such that when n is 1 then ring T is a Six membered ring, and when n is 2 then ring T is a seven membered ring;
   (B) R¹ is selected from the group consisting of:
      (1) H;
      (2) C₁ to C₆ alkyl:
      (3) allyl; and
      (4) propargyl;
   (C) R³ and R⁴ are independently selected from the group consisting of:
      (1) H;
      (2) C₁ to C₆ alkyl;
      (3) allyl;
      (4) propargyl; and
      (5) —(CH₂)_{q}—R⁵ wherein q is an integer of: 1 to 7, and R⁵ is selected from the group consisting of: phenyl, substituted phenyl, —OR⁶, —C(O)OR⁶, —C(O)R⁶, —OC(O)R⁶, —C(O)NR⁶R⁷, CN and —SR⁶ wherein R⁶ and R⁷ are as defined below, and wherein the substituents on said substituted phenyl are each independently selected from the group consisting of: —OH, —O—(C₁ to C₆)alkyl, halogen, C₁ to C₆ alkyl, —CF₃, —CN, and NO₂, and wherein said substituted phenyl contains from 1 to 3 substituents;
   (D) R⁶ and R⁷ are each independently selected from the group consisting of: H and C₁ to C₆ allyl; and
   (E) R³ and R⁴ are each independently bound to the same or different carbon atom of ring T.

U.S. Pat. No. 6,034,251 discloses compounds comprising the following structural formula: 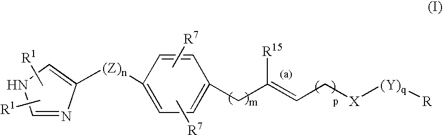
or a pharmaceutically acceptable salt or solvate thereof, wherein:
   the double bond (a) is E or Z (that is the double bond to the carbon atom having the R¹⁵ substituent is of the E or Z configuration);
      each R¹ is independently selected from the group consisting of hydrogen, lower alkyl, trihalomethyl, phenyl and benzyl;
      each R⁷ is independently selected from the group consisting of hydrogen, lower alkyl, halogen, trihalomethyl, NR¹⁰R¹¹, or a group OR¹⁰, whereby R¹⁰ and R¹¹ are independently selected from hydrogen, lower alkyl or trihalomethyl;
      X is —CONR⁵—; —SO₂—, —S—; —CO—; —COO—; —CN(OR⁵)NR⁵—; —C(NR⁵)NR⁵—; —SONR⁵—; —SO₂NR⁵— and, provided p is not zero, X may also be —O—; —NR⁵—; —NR⁵CONR⁵—; —OCONR⁵—; —O—CO— or —NR⁵CO—;
      Y is C₁-C₃-alkyl, optionally substituted at any carbon atom of the group by one substituent R⁵;
      Z is C(R¹)₂; wherein no more than two R¹ groups are other than hydrogen;
      n is 1 or 2;
      m is 0 or 1;
      p is 0 or 1;
      q is 0 or 1;
      R is selected from C₃ to C₇ cycloalkyl, heterocyclic groups, aryl or heteroaryl, wherein said R groups are optionally substituted with 1-3 substituents as defined below;
      each R⁵ independently represents hydrogen, lower alkyl or poly-haloloweralkyl; and
      R¹⁵ represents H or lower alkyl (e.g., methyl). U.S. Pat. No. 6,100,279 discloses compounds comprising the following structural formula: 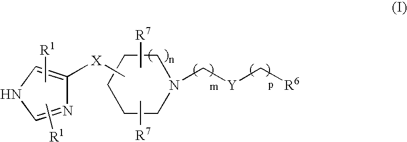
or pharmaceutically acceptable salts or solvate thereof, wherein:
   X is a straight chain alkyl group having 1 to 7 carbon atoms or an alkene or alkyne group with 2 to 4 carbon atoms; wherein said alkyl or alkene groups are optionally substituted with up to two (i.e., 1 or 2) R⁷ groups;
   n is 0, 1 or 2,
   m and p are 0 to 4;
   when m is 0 to 4, Y represents —SO₂—; —CS—; —CO—; —CONR⁵—; —CO(CH₂)_{w}O— (with w being 1 to 4); —COO—; —CON(OR⁵)—; —C(NR⁵)NR⁵—; —SO₂NR⁵— or —CSNR⁵—;
   when m is 2 to 4, Y represents all the groups above when m is 0 to 4 and, in addition, Y represent —CHOR⁵—; —O—; —NR⁵CONR⁵—; —NR⁵CO—; —NR⁵—; —OCONR⁵—; —NR⁵C(NR⁵)NR⁵—; —NR⁵CSNR⁵; —NR⁵CS— or —NR⁵SO₂—; —NR⁵C(O)O—; or —CSNR⁵—;
   each R⁵ independently represents hydrogen, alkyl or benzyl;
   R⁶ represents aryl, heteroaryl, or 3- to 7-membered heterocyclic group having one to three heteroatoms in the ring, wherein the heteroatoms are selected from N, S and O, and wherein said R⁶ group is optionally substituted by one to three substituents as defined below;
   when Y is —SO₂—, then R⁶, in addition to the above groups, also represents alkyl having 1 to 7 carbon atoms or a group-NR¹⁰R¹¹ wherein R¹⁰ and R¹¹ are independently selected from H, alkyl or trihalomethyl;
   each R¹ is independently hydrogen, alkyl or trihalomethyl;
   each R⁷ is independently selected from hydrogen, alkyl, trihalomethyl, phenyl or benzyl, wherein said phenyl and benzyl are optionally substituted by one to three substituents independently selected from of alkyl, halogen, trihalomethyl, CN, NO₂, OR¹⁰ or NR¹⁰R¹¹, wherein R¹⁰ and R¹¹ are as above defined.

U.S. Pat. No. 5,578,616 discloses compounds comprising the following structural formula: 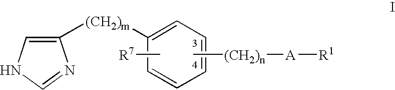
wherein:
   A is selected from —O—CO—NR¹—, —CO—, —NR¹—CO—NR¹—, —NR¹—CO—, —NR¹—, —O—, —CO—NR¹—, —CO—O—, and —C(:NR¹)—NR¹—;
   the groups R¹, which may be the same or different when there are two or three such groups in the molecule of formula I, are selected from hydrogen, and lower alkyl, aryl, cycloalkyl, heterocyclic and heterocyclyl-alkyl groups, and groups of the formula —(CH₂)_{y}-G, where G is selected from CO₂R³, COR³, CONR³R⁴, OR³, SR³, NR³R⁴, heteroaryl and phenyl, which phenyl is optionally substituted by halogen, lower alkoxy or polyhaloloweralkyl, and y is an integer from 1 to 3;
   R² is selected from hydrogen and halogen atoms, and alkyl, alkenyl, alkynyl and trifluoromethyl groups, and groups of the formula OR³, SR³ and NR³R⁴;
   R³ and R⁴ are independently selected from hydrogen, and lower alkyl and cycloalkyl groups, or R³ and R⁴ together with the intervening nitrogen atom can form a saturated ring containing 4 to 6 carbon atoms that can be substituted with one or two lower alkyl groups;
   with the proviso that, when y is 1 and G is OR³, SR³ or NR³R⁴, then neither R³ nor R⁴ is hydrogen;
   the group —(CH₂)ₙ-A-R¹ is at the 3- or 4-position, and the group R² is at any free position;
   m is an integer from 1 to 3;
   and n is 0 or an integer from 1 to 3;
   or a pharmaceutically acceptable acid addition salt thereof;
   or a pharmaceutically acceptable salt thereof with a base when G is CO₂H;
including a tautomeric form thereof.

U.S. Pat. No. 5,990,147 discloses compounds comprising the following structural formula: 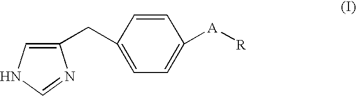
or pharmaceutically acceptable acid addition salt or solvate thereof (or tautomer thereof, wherein;
   A is —CH₂—NH—CO—NH—; —CH₂—O—CO—NH— or —CH₂CH₂—CO—NH—(CH₂)ₘ—;
   m is 0, 1 or 2;
   R is the group 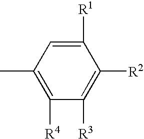
wherein at least two of R¹, R², R³ and R⁴ are hydrogen and the two others are independently selected from H, halogen (e.g., Br, I, F, or Cl), CH₃, CF₃, OCH₃, OCF₃ or CN; and
   with the proviso, that when A is —CH₂—O—CO—NH— and R¹, R³ and R⁴ are all hydrogen, then R² can not be Cl.

U.S. Pat. No. 5,807,872 discloses compounds comprising the following structural formula: 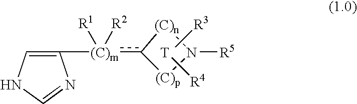
or a pharmaceutically acceptable salt or solvate thereof, wherein:
   (A) in is an integer selected from the group consisting of: 1 and 2;
   (B) n and p are integers and are each independently selected from the group consisting of: 0, 1, 2, 3 and 4 such that the sum of n and p is 4 and T is a 6-membered ring;
   (C) R³ and R⁴ are each independently bound to the same or different carbon atom of ring T such that there is only one R³ group and one R⁴ group in ring T, and each R¹, R², R³, and R⁴ is independently selected from the group consisting of:
      (1) H;
      (2) C¹ to C⁶ alkyl; and
      (3) —(CH₂)_{q}—R₆ wherein q is an integer of: 1 to 7, and R⁶ is selected from the group consisting of: phenyl, substituted phenyl, —OR⁷, —C(O)OR⁷, —C(O)R⁷, —OC(O)R⁷, —C(O)NR⁷R⁸, CN and —SR⁷ wherein R⁷ and R⁸ are as defined below, and wherein the substituents on said substituted phenyl are each independently selected from the group consisting of: —OH, —O—(C₁ to C₆)alkyl, halogen, C₁ to C₆ alkyl, —CF₃, —CN, and —NO₂, and wherein said substituted phenyl contains from 1 to 3 substituents;
   (D) R⁵ is selected from the group consisting of:
      (1) H;
      (2) C₁ to C₂₀ alkyl;
      (3) C₃ to C₆ cycloalkyl;
      (4) —C(O)OR^{7′}; wherein R^{7′} is the same as R⁷ defined below except that R^{7′} is not H;
      (5) C(O)R⁷;
      (6) C(O)NR⁷R⁸;
      (7) allyl;
      (8) propargyl; and
      (9) —(CH₂)_{q}—R⁶ wherein q and R⁶ are as defined above, and when q is equal to 1, then R⁶ is not OH or SH;
   (E) R⁷ and R⁸ are each independently selected from the group consisting of: H, C₆ to C₆ alkyl, and C₃ to C₆ cycloalkyl;
   (F) the dotted line (------) represents a double bond that is optionally present when m is 1, and n is not 0, and p is not 0 (ire., the nitrogen in the ring is not bound directly to the carbon atom bearing the double bond), and when said double bond is present then R² is absent; and
   (G) when m is 2, each R¹ is the same or different substituent for each m, and each R² is the same or different substituent for each m, and at least two of the substituents R¹ and/or R² are H.

Those skilled in the art will appreciate that the total number of substituents on each of the —(C)ₙ— and —(C)ₚ— groups is two, and that such substituents are independently selected from the group consisting of hydrogen, R³ and R⁴, such that there is a total of only one R³ and one R⁴ substituent in ring T.

The following PCT Publication discloses H3 antagonists and H1/H3 dual antagonists which may be used in the present invention: PCT Publication No. WO 02/24658 discloses compounds comprising the following formula: 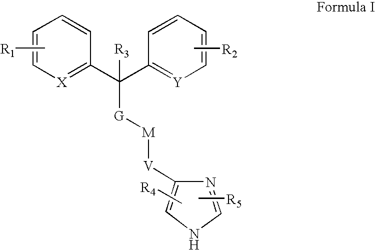
wherein
G is selected from the group consisting of C₁-C₆ alkyl or a bond;
M is a moiety selected from the group consisting of —C≡C—, —C≡C—, —C(═NR⁷)—NR⁶—, —NR⁶—C(═NR⁷), —NR⁶—C(O)—NR⁶—, —NR⁶—C(O)—O—, —O—C(O)—NR⁶—, —NR⁶—C(O)—, —C(O)—NR⁶—, —O—, —NR⁶—, —C(O)—, —N⁺R⁶R⁸—, and 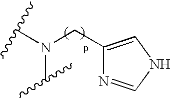
p is 1-6
V is C₁-C₆ alkyl;
X and Y may be the same or different and are independently selected from the group consisting of N, CH, or N-oxide, with the proviso that at least one of X and
Y is N or N-oxide;
R¹ and R² may each number 1-4 and are independently selected from the group consisting of hydrogen, lower alkyl, lower alkoxy, halogen, polyhalolower alkyl, —OH, —N(R⁶)₂, —NO₂, —CN, —COOR⁶, —CONR⁶R⁶, and —NR⁶—C(O)—R⁷ (wherein R⁷ is not —OH or —CN);
R³ is selected from hydrogen, lower alkyl, lower alkoxy, hydroxyl, polyhalolower alkyl, and a bond forming a double bond towards the moiety G when G is C₁-C₆ alkyl;
R⁴ and R⁵ are independently selected from the group consisting of hydrogen, lower alkyl, and polyhalolower alkyl;
R⁶ and R⁸ are independently selected from hydrogen, lower alkyl, aralkyl, alkylaryl, polyhalolower alkyl, substituted or unsubstituted phenyl; and substituted or unsubstituted benzyl; and
R⁷ is selected from H, OH, alkoxy, cyano, phenyl, substituted phenyl, benzyl, and substituted benzyl;
with the proviso that when G is a bond and when M is either —O— or —O—C(O)—NR⁶—, then one of X and Y is N; and with the further proviso that when R³ is —OH or alkoxyl, and G is a bond, then M≠O or NR⁶.

PCT Publication No. WO 02/24659 discloses compounds comprising the following structural formula: 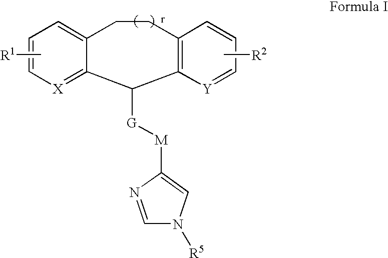
wherein:
   [symbol] =0, 1 or 2;
   X and Y are independently selected from the group consisting of N, CH or N-oxide;
   G is a moiety selected from the group consisting of the moieties II, III and IV with the top and of said II, III and IV being linked to the tricyclic moiety and the bottom end of II, III and IV being linked to M: 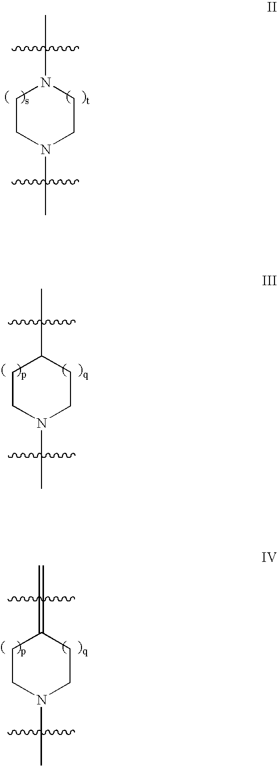
   where [symbol] = [symbol] =1 or 2; and p=q=0, 1 or 2;
   M is a moiety selected from the group consisting of C₁-C₆alkyl;
   —C(O)—(CH₂) —; —(CH₂) -A-(CH₂) —; —C(O)—O—(CH ) —; and —C(O)—NR²—(CH₂) —; where A [symbol] O, S(O) —; and —NR⁴—;
   n=0, 1, 2 or 3;
   x is a whole number in the range 2-5;
   y is a whole number in the range 0-5;
   [symbol] is a number in the range 0-5;
   [symbol] =0, 1, or 2;
   R¹ and R² may each number 1-3 and are independently selected from the group consisting of hydrogen, lower alkyl, lower alkoxy, halogen, OCF , OCHF₂, —OH, and —N(R )₂;
   R³ is selected from the group consisting of hydrogen, lower alkyl, and polyhaloloweralkyl;
   R is selected from hydrogen, lower alkyl, polyhalolower alkyl, and
   R is H, C₁-C alkyl or OH.

PCT Publication No. WO 02/44141 discloses compounds comprising the following structural formula: 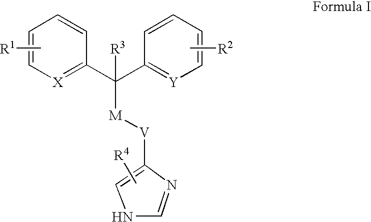
M is a moiety having a general structure shown in Formula II or III: 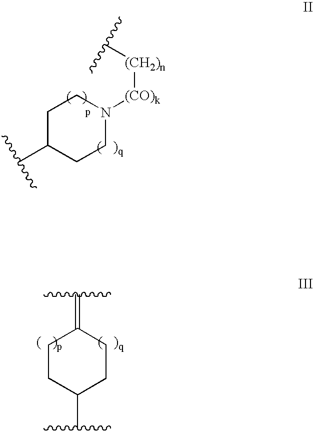
where k=0 or 1, n=0-5, and p=q=0, 1 or 2 with the proviso that when M is Formula III, R³ is absent;
V is a moiety selected from the group consisting of C₁-C₆ alkyl; —(CH₂)ₓ-A-(CH₂)_{y}—; and —(CH₂)_{c}-A-(CH₂)ₘ—C(O)—N(R⁷)—(CH₂)_{d}—, where A is —O—, —S(O)ᵣ—, and —NR⁷—; m=0, 1, 2 or 3; x is a whole number in the range 28; y is a whole number in the range 1-5; c is a whole number in the range 2-4; and r=0, 1 or 2; d is a number in the range 0-5;
   X and Y are independently selected from the group consisting of N, CH, and N(O);
   Z is selected from the group consisting of N, CH and N(O);
   R¹ and R² may each number 1-4 and are independently selected from the group consisting of hydrogen, lower alkyl, lower alkoxy, halogen, polyhalolower alkyl, polyhalolower alkoxy, —OH, CN, NO₂, or COOR⁸;
   R³ is selected from hydrogen, lower alkyl, lower alkoxy, hydroxyl, with the proviso that when n and k are both 0, then R³ is not —OH or alkoxy;
   R⁴ is selected from the group consisting of hydrogen, lower alkyl, polyhalolower alkyl or —OH; and
   R⁷ and R⁸ are independently selected from hydrogen, lower alkyl, substituted or unsubstituted phenyl; and substituted or unsubstituted benzyl.

PCT Publication No. WO 02/24657 discloses compounds comprising the following structural formula: 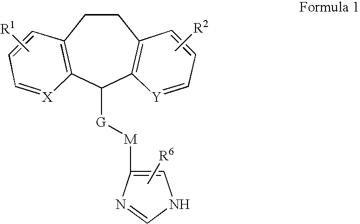
wherein
G is selected from the group consisting of —(CH₂)ᵥ—NR³—, —(CH₂)ᵥ—O—, (CH₂)ᵥ—S(O)_{z}—, —(CH₂)ᵥ—NR³—C(NR⁴)—NR³—, —(CH₂)ᵥ—O—C(O)NR³—, —(CH₂)ᵥ—NR³C(O)NR³—, —(CH₂)ᵥ—NR³C(O)—, —(CH₂)ᵥ—NR³C(O)—, —(CH₂)ᵥC(O)NR⁵—;
M is a branched or unbranched alkyl group consisting of 1-6 carbon atoms, or a branched or unbranched alkenyl group consisting of 2-6 carbon atoms;
X and Y are Independently selected from the group consisting of N, CH or N-oxide:
R¹ and R² may each number 1-4 and are independently selected from the group consisting of H, halogen, lower alkyl, lower alkoxy, polyhalo lower alkoxy, OH, CF₃, NH₂, NHC(O)alkyl, CN or NO₂;
   R³ is independently selected from the group consisting of H, lower alkyl, substituted or unsubstituted phenyl, substituted or unsubstituted benzyl, or a group of the formula: 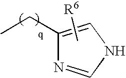
   R⁴ is selected from the group consisting of H, CN, CO₂R⁵;
   R⁵ is selected from the group consisting of lower alkyl and substituted or unsubstituted benzyl;
   R⁶ is selected from the group consisting of H or lower alkyl;
   q is 2-5;
   v is 0-6; and
   z is 0, 1 or 2.

PCT Publication No. WO 02/72548 discloses compounds comprising the following structural formula: 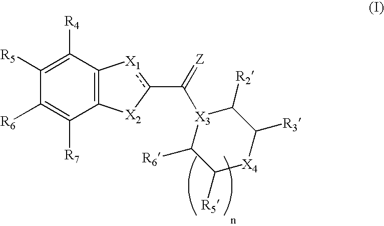
Wherein R₁ is Rₐ, RₐR_{b}, —Rₐ—O—R_{b}—, or (R_{c})(R_{d})N—R_{b}—, where Rₐ is H, cyano, —(C═O)N(R_{c})(R_{d}), —C(═NH)(NH₂), C₁₋₁₀ alkyl, C₃₋₈ alkenyl, C₃₋₈ cycloalkyl, C₂₋₅, heterocyclic radical, or phenyl; where R_{b} is C₁₋₈ alkylene, C₂₋₈ alkenylene, C₃₋₈ cycloalkylene, bivalent C₃₋₈ heterocyclic radical, or phenylene; and R_{c} and R_{d} are each independently H, C₁₋₈ alkyl, C₂₋₈alkenyl, C₃₋₈ cycloalkyl, or phenyl;
R_{2′} is H, methyl, ethyl, NRₚR_{q}, —(CO)NRₚR_{q}, —(CO)ORᵣ, —CH₂NRₚR_{q}, or CH₂ORᵣ; where Rₚ, R_{q}, and Rᵣ are independently selected from C₁₋₆ alkyl, C₃₋₆ cycloalkyl, phenyl; (C₃₋₆ cycloalkyl)(C₁₋₂ alkylene), benzyl or phenethyl; or Rₚ and R_{q} taken together with the nitrogen to which they are attached, form a 4-7 membered heterocyclic ring with 0 or 1 additional heteroatoms selected from O, S, and N;
R_{3′} is H, methyl, ethyl, NRₛRₜ, —(CO)NRₛRₜ, —(CO)ORᵤ, —CH₂NRₛRₜ, or CH₂ORᵤ; where Rₛ, Rₜ, and Rᵤ are independently selected from C₁₋₈ alkyl, C₃₋₆ cycloalkyl, phenyl; (C₃₋₆ cycloalkyl)(C₁₋₂ alkylene), benzyl or phenethyl; or Rₛ and Rₜ taken together with the nitrogen to which they are attached, form a 4-7 membered heterocyclic ring with 0 or 1 additional heteroatoms selected from O, S, and N;
R_{5′} is methyl, ethyl, or H;
R_{6′} is methyl, ethyl, or H;
R^{7′} is methyl, ethyl, or H;
   X₄ is NR₁ or S;
   X₁ is CR₃;
   R₃ is F, Cl, Br, CHO, R_{f}, R_{f}R_{g}—, R_{f}—O—R_{g}—, or (Rₕ)(Rᵢ)N—R_{g}—, where R_{f} is H, C₁₋₆ alkyl, C₂₋₆ alkenyl, C₃₋₆ cycloalkyl, C₂₋₅ heterocyclic radical, or phenyl; where R_{g} is C₁₋₆ alkylene, C₂₋₆ alkenylene, C₃₋₆ cycloalkylene, bivalent C₃₋₆ heterocyclic radical, or phenylene; and Rₕ and Rᵢ are each independently H, C₁₋₆ alkyl, C₂₋₆ alkenyl, C₃₋₆ cycloalkyl, or phenyl;
   X₂ is NR or O, provided that X₂ is NR where X₁ is N; R is H or C₁₋₆ alkyl;
   X₃ is N;
   Z is ═O or ═S;
   each of R₄ and R₆ is independently H, F, Cl, Br, I, COOH, OH, nitro, amino, cyano, C₁₋₄ alkoxy, or C₁₋₄ alkyl;
   R₅ is H, F, Cl, Br, I, (C═O)Rⱼ, OH, nitro, NRⱼRₖ, cyano, phenyl, —OCH₂—Ph, C₁₋₄ alkoxy, or C₁₋₄ alkyl;
   R₇ is H, F, Cl, Br, I, (C═O)Rₘ, OH, nitro, NRᵢRₘ, cyano, phenyl, —OCH₂—PhC₁₋₄ alkoxy, or C₁₋₄ alkyl;
   wherein each of Rⱼ, Rₖ, Rᵢ, and Rₘ is independently selected from H, C₁₋₆ alkyl, hydroxy, phenyl, benzyl, phenethyl, and C₁₋₆ alkoxy;
   each of the above hydrocarbyl (including alkyl, alkoxy, phenyl, benzyl, cycloalkyl, and so on) or heterocyclic groups being independently and optionally substituted with between 1 and 3 substituents selected from C₁₋₃ alkyl, halo, hydroxy, amino, and C₁₋₃ alkoxy;
   wherein n is 0, 1, or 2; where n is 2, the moiety —(CHR_{5′})ₙ₌₂— is —(CHR_{5′}—CHR_{7′})— where CHR_{6′} is between CHR_{6′} and CHR_{7′};
   provided at least one of R₁, R₂, R₃, R₄, R₅, R₉, and R₇ is other than H when Z is O;
   and provided, where Z is O, n=1, and each of R₄, R₅, R₆, R₇, R_{2′}, R_{3′}, R_{5′}, and R_{6′} is H, (or at least 7, 8, or 9 of these 10 limitations apply) then (a) where X₂ is NH, then R₁ is (i) not methyl, pyridyl, phenyl, or benzyl, or (ii) is selected from the disclosed possibilities, but not C₁₋₂ alkyl and not a six-membered aryl or six-membered nitrogen-containing heteroaryl, or phenyl(C₁₋₂ alkylene) (alternatively, provided, where Z is O, n=1, and X₂ is NH, then at least two (or three) of R₄, R₅, R₆, R₇, R_{2′}, R_{3′}, R^{5′}, and R^{6′} is other than H); and (b) where X₂ is O, then R₁ is not methyl;
   and provided, where Z is O, X₂ is NH, n=1, R₁ is methyl, each of R₄, R₆, R₇, R_{2′}, R_{3′}, R_{5′}, and R_{6′}, is H (or at least 7, 8, 9, or 10 of these 11 limitations apply), then R₅ is (i) not methoxy, (ii) not methoxy, or ethoxy, (iii) not C₁₋₄ alkoxy, or (iv) not methoxy or hydroxy;
   or a pharmaceutically acceptable salt, ester, or amide thereof.

According to one aspect of the invention, the invention features compounds of the following formula (Ib): 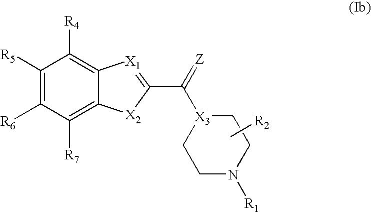
Wherein R₁ is Rₐ, RₐR_{b}—, Rₐ—O—R_{b}—, or (R_{c})(R_{d})N—R_{b}—, where Rₐ is H, C₁₋₁₀ alkyl, C₃₋₈ alkenyl, C₃₋₈ cycloalkyl, C₂₋₅ heterocyclic radical, or phenyl; where R_{b} is C₁₋₈ alkylene, C₃₋₈ alkenylene, C₃₋₈ cycloalkylene, bivalent C₃₋₈ heterocyclic radical, or phenylene; and R_{c} and R_{d} are each independently H, C₁₋₈ alkyl, C₃₋₈ alkenyl, C₃₋₈ cycloalkyl, or phenyl;
R₂ is ortho (like R_{2′} in formula (I)) or meta (like R_{3′} in formula (I)), and is methyl or H;
X₁ is CR₃;
R₃ is F, Cl, Br, R_{f}, R_{f}R_{g}, R_{f}—O—R_{g}—, or (Rₕ)(Rᵢ)N—R_{g}—, where R_{f} is H, C₁₋₆ alkyl, C₂₋₆ alkenyl, C₃₋₆cycloalkyl, C₂₋₅ heterocyclic radical, or phenyl; where R_{g} is C₁₋₆ alkylene, C₂₋₆ alkenylene, C₃₋₈ cycloalkylene, bivalent C₃₋₆ heterocyclic radical, or phenylene; and Rₕ and Rⁱ are each independently H, C₁₋₆ alkyl, C₂₋₆ alkenyl, C₃₋₈ cycloalkyl, or phenyl;
X₂ is NRₑ or O, provided that X₂ is NRₑ, when X₁ is N; Rₑ is H or C₁₋₆ alkyl;
X₃ is N;
Z is ═O or ═S;
each of R₄ and R₆ is independently H, F, Cl, Br, I, COOH, OH, nitro, amino, cyano, C₁₋₄ alkoxy, or C₁₋₄ alkyl;
R₅ is H, F, Cl, Br, I, (C+O)Rⱼ, OH, nitro, NRⱼRₖ, cyano, —OCH₂—Ph, C₁₋₄ alkoxy, or C₁₋₄ alkyl;
R₇ is H, F, Cl, Br, I, (C═O)Rₘ, OH, nitro, NRₗRₘ, cyano, C₁₋₄ alkoxy, or C₁₋₄ alkyl;
wherein each of Rⱼ, Rₖ, Rₗ, and Rₘ is independently selected from H, C₁₋₆ alkyl, hydroxy, and C₁₋₆ alkoxy; and
each of the above hydrocarbyl or heterocyclic groups being independently and optionally substituted with between 1 and 3 substituents selected from C₁₋₃ alkyl, halo, hydroxy, amino, and C₁₋₃ alkoxy;
provided at least one of R₁, R₂, R₃, R₄, R₅, R₆, and R₇ is other than H when Z is ═O;
   or a pharmaceutically acceptable salt, ester or amide thereof.

The present invention comprises compositions comprising an antagonist or a combination of antagonists which antagonize the H3 receptor (e.g., any of the H3 antagonists mentioned herein), the H4 receptor (e.g., any of the H4 antagonists mentioned herein), and, optionally, the H1 receptor (e.g., any of the H1 antagonists mentioned herein) and pharmaceutical compositions thereof.

The ability of any substance to bind to a histamine receptor can be evaluated by using the methods set forth herein (e.g., Examples) or by using the examples set forth in U.S. Pat. No. 6,204,017.

Other antagonists may be, for example, small molecules, nucleic acids (e.g., antisense oligonucleotides which bind to H1, H3 or H4 histamine receptor mRNA), peptides, or antibodies (and antigen-binding fragments thereof) which bind specifically to an H1, H3 or H4 receptor.

### Pharmaceutical Compositions, Dosage and Administration

The present invention also includes a pharmaceutical composition comprising a histamine H3 receptor antagonist, a histamine H4 receptor antagonist and, optionally, a histamine H1 receptor antagonist along with a pharmaceutically acceptable carrier along with methods for administrating the compositions to treat allergic conditions. The pharmaceutical compositions may be prepared by any methods well known in the art of pharmacy; see, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

Pharmaceutical compositions containing the antagonists can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intramuscular, intraperitoneal, intravenous), and non-parenteral (e.g., topical, ocular, transdermal, sublingual, inhalation, rectal, oral).

Unit forms of administration include oral forms such as tablets, capsules, powders, cachets, granules and solutions or suspensions, sublingual and buccal forms of administration, aerosols, implants, subcutaneous, intramuscular, intravenous, intranasal, intraocular, subcutaneous or rectal forms of administration.

When a solid composition is prepared in the form of tablets, e.g., a wetting agent such as sodium lauryl sulfate can be added to micronized or non-micronized antagonists and mixed with a pharmaceutical vehicle such as silica, gelatin starch, lactose, magnesium stearate, talc, gum arabic or the like. The tablets can be coated with sucrose, various polymers, or other appropriate substances. Tablets can be treated so as to have a prolonged or delayed activity and so as to release a predetermined amount of active principle continuously or at predetermined intervals, e.g., by using ionic resins and the like.

A preparation in the form of gelatin capsules may be obtained, e.g., by mixing the antagonists with a diluent, such as a glycol or a glycerol ester, and incorporating the resulting mixture into soft or hard gelatin capsules.

A preparation in the form of a syrup or elixir can contain the antagonists together, e.g., with a sweetener, methylparaben and propylparaben as antiseptics, flavoring agents and an appropriate color.

Water-dispersible powders or granules can contain the antagonists mixed, e.g., with dispersants, wetting agents or suspending agents, such as polyvinylpyrrolidone, as well as with sweeteners and/or other flavoring agents.

Rectal administration may be provided by using suppositories which may be prepared, e.g., with binders melting at the rectal temperature, for example cocoa butter or polyethylene glycols.

Parenteral, intranasal or intraocular administration may be provided by using, e.g., aqueous suspensions, isotonic saline solutions or sterile and injectable solutions containing pharmacologically compatible dispersants and/or solubilizers, for example, propylene glycol or polyethylene glycol.

Thus, to prepare an aqueous solution for intravenous injection, it is possible to use a co-solvent, e.g., an alcohol such as ethanol or a glycol such as polyethylene glycol or propylene glycol, and a hydrophilic surfactant such as Tween® 80. An oily, intramuscular injectable solution can be prepared, e.g., by solubilizing the antagonists with a triglyceride or a glycerol ester.

Topical administration can be provided by using, e.g., creams, ointments or gels.

Transdermal administration can be provided by using patches in the form of a multilaminate, or with a reservoir, containing the antagonists and an appropriate solvent.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing the antagonists, by themselves or associated with an excipient, in powder form.

The antagonists can also be formulated as microcapsules or microspheres, e.g., liposomes, optionally with one or more carriers or additives.

Implants are among the prolonged release forms which can be used in the case of chronic treatments. They can be prepared in the form of an oily suspension or in the form of a suspension of microspheres in an isotonic medium.

The daily dose of a antagonists can be determined by a clinician and is generally dependent on the potency of the compound administered, the age, weight, condition and response of the subject.

Methods of the present invention may include administration of the antagonists along with, for example, known antihistamine, decongestant or anti-allergy agents. The administration and dosage of such agents is typically as according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art. For example, histamine antagonists of the present invention can be administered to a patient at a “therapeutically effective dosage”. A therapeutically effective dosage is any dosage which is sufficient to alleviate or prevent the symptoms or physiological effects of allergic or non-allergic airway obstruction including, but not limited to, allergic rhinitis, congestion (e.g., sinus congestion), pulmonary inflammation, acute respiratory distress syndrome, asthma, bronchitis, chronic obstructive pulmonary disease, pulmonary fibrosis, emphysema, respiratory infections and sinus infections to any degree. In one embodiment of the invention, a histamine receptor antagonist of the present invention is administered to a patient or subject in need of such treatment (e.g., a patient or subject suffering from or susceptible to any of the indications mentioned herein) at a dosage of about 5 to about 2000 mg per day or about 50 mg per day to about 1900 mg/day or about 100 mg per day to about 1800 mg/day or about 300 mg per day to about 1600 mg/day or about 500 mg per day to about 1200 mg/day or about 750 mg per day to about 1000 mg/day or about 5 mg per day to about 500 mg per day or about 500 mg per day to about 1000 mg per day or about 1000 mg per day to about 2000 mg per day.

Typical agents which may be included along with the histamine receptor antagonists include glucocorticoids (e.g., mometasone, fluticasone, budesonide), Non-steroidal anti-inflammatory drugs (NSAIDs) (e.g., COX2 inhibitors (e.g, rofecoxib, celecoxib) ibuprofen, naproxen), leukotriene receptor antagonists (e.g., montelukast sodium), M3 antagonists (e.g., ipratropium, tiotropium) and antibiotics (e.g., penicillin, amoxicillin, ampicillin, methicillin).

The histamine receptor antagonists of the invention along with any additional agents (discussed above) may be formulated together into a single composition or into two or more separate compositions for simultaneous consumption. Alternatively, for example, an H1 antagonists may be administered to a subject at a different time than when the H3 and H4 antagonists are administered; for example, each administration may be given non-simultaneously at several intervals over a given period of time.

### Indications

The compositions of the present invention can be used to treat or prevent medical conditions characterized by allergic or non-allergic airway obstruction including, but not limited to, allergic rhinitis, congestion (e.g., sinus congestion), pulmonary inflammation, acute respiratory distress syndrome, asthma, bronchitis, chronic obstructive pulmonary disease, pulmonary fibrosis, emphysema, respiratory infections and sinus infections.

Clinical symptoms of seasonal allergic rhinitis typically include nasal itching and irritation, sneezing and watery rhinorrhea, frequently accompanied by nasal congestion. The perennial allergic rhinitis clinical symptoms are similar, except that nasal blockage may be more pronounced. Either type of allergic rhinitis may also cause other symptoms such as itching of the throat and/or eyes, epiphora and edema around the eyes. These symptoms may vary in intensity from the nuisance level to debilitating. Other types of rhinitis present similar symptoms. In addition to other processes, allergic rhinitis involves the release of histamine (e.g., from mast cells) which is a mediator in immediate hypersensitivity reactions.

Congestion, particularly sinus congestion, involves blockage of one or more of the four pairs of sinus passageways in the skull. The blockage may result from inflammation and swelling of the nasal tissues or from secretion of mucus. It may be acute or chronic. Acute sinus congestion is most often caused by the common cold. Chronic sinus congestion may result from environmental irritants such as tobacco smoke, food allergens, inhaled allergens, or foreign bodies in the nose. Sinus congestion leads to impaired flow of fluids in the sinuses, which predisposes individuals to bacterial infections that can cause sinusitis.

Pulmonary inflammation is a condition which is often characterized by wheezing and shortness of breath. When the lungs are exposed to allergens (e.g., particulate matter, automobile exhaust or pollen) and pathogens (e.g., Pseudomonas aeruginosa) pulmonary inflammation often occurs.

Adult (acute) respiratory distress syndrome (ARDS) is a condition characterized by pulmonary inflammation. In general, ARDS results in the rapid onset of progressive malfunction of the lungs, especially with regard to the ability to take in oxygen, usually associated with the malfunction of other organs. The condition is associated with extensive pulmonary inflammation and small blood vessel injury in all affected organs.

The fundamental pathophysiologic entity resulting in the clinical disease of asthma is airway inflammation. Histological findings in the asthmatic airway may include bronchial occlusion with mucous and cellular debris, denudation of the epithelial layer, edema and inflammatory infiltrate in the submucosa, mucous gland hypertrophy, and bronchial smooth muscle hypertrophy.

The methods and compositions of the present invention may also be used to treat chronic bronchitis, chronic obstructive pulmonary disease (COPD), pulmonary fibrosis, emphysema and sinus and respiratory infections.

### Kits

The present invention also provides kits comprising the components of the combinations of the invention in kit form. A kit of the present invention includes one or more components including, but not limited to, one or more histamine H3 antagonists, for example, as discussed herein, in association with one or more histamine H4 receptor antagonists, for example, as discussed herein and, optionally, in association with one or more histamine H1 receptor, for example, as discussed herein. The antagonists can be formulated as a pure composition or in combination with a pharmaceutically acceptable carrier, in a pharmaceutical composition.

In one embodiment, a kit includes one or more histamine H3 antagonists, or a pharmaceutical composition thereof, in one container (e.g., in a sterile glass or plastic vial), one or more histamine H4 antagonists, or a pharmaceutical composition thereof, in another container (e.g., in a sterile glass or plastic vial) and, optionally, one or more histamine H1 antagonists, or a pharmaceutical composition thereof, in another container (e.g., in a sterile glass or plastic vial).

In another embodiment of the invention, the kit comprises a combination of the invention, including one or more histamine H3 antagonists along with one or more histamine H4 antagonists and, optionally, one or more histamine H1 antagonists formulated together, optionally, along with a pharmaceutically acceptable carrier, in a pharmaceutical composition, in a single, common container.

If the kit includes a pharmaceutical composition for parenteral administration to a subject, the kit can include a device for performing such administration. For example, the kit can include one or more hypodermic needles or other injection devices.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

The following examples are provided to further describe the present invention and should not be construed to limit the scope of the present invention.

The following examples make reference to standard methods known to those skilled in the art which may be performed, as described, e.g., in Maniatis, et al., Molecular Cloning: A Laboratory Manual, 1982, Cold Spring Harbor Laboratory, Cold Spring Harbor Press; Sambrook, et al., Molecular Cloning: A Laboratory Manual, (2d ed.), Vols 1-3, 1989, Cold Spring Harbor Press, NY; Ausubel, et al., Biology, Greene Publishing Associates, Brooklyn, N.Y.; or Ausubel, et al., (1987 and Supplements), Current Protocols in Molecular Biology, Greene/Wiley, New York; Innis, et al., (eds.) PCR Protocols: A Guide to Methods and Applications, 1990, Academic Press, N.Y.

### Example 1

### Screening Assays for Histamine H4 Receptor Antagonists

In this example, the ability of several compounds to compete against radiolabeled histamine for binding to membrane-bound human histamine H4 receptor is evaluated. The histamine H4 receptor used in this assay is SP9144 which is set forth in SEQ ID NOs: 1 and 2 of U.S. Pat. No. 6,204,017.

Membrane preparation. Forty-eight hours after transfection with plasmid containing the sequence for SP9144, HEK293 cells were harvested from T150 flasks by incubating 5 minutes in 5 ml of 5 mM EDTA/Hanks' balanced salt solution followed by repeated pipeting. They were centrifuged 5 minutes at 1000×g. The EDTA/PBS was decanted and an equal volume of ice-cold 50 mM Tris-HCl, pH 7.5, was added and cells were broken up with a Polytron (PT10 tip, setting 5, 30 seconds). Nuclei and unbroken cells were sedimented at 1000×g for 10 minutes and then the supernatant was centrifuged at 50,000×g for 10 minutes. The supernatant was decanted, the pellet was resuspended by Polytron, a sample was taken for protein assay (bicinchoninic acid, Pierce; Rockford, Ill.), and the tissue was again centrifuged at 50,000×g. Pellets were stored frozen at −20° C.

Binding assay. For saturation binding, four concentrations of [³H]-histamine (15 Ci/mmol, Dupont NEN; Boston, Mass.) were incubated without and with 10⁻⁵ M histamine in triplicate with 50 μg of membrane protein in a total volume of 200 μl of 50 mM Tris-HCl, pH 7.5, for 30 minutes at 30° C. Samples were filtered on GF/B filters and washed thrice with 2 ml of cold Tris buffer. Filters were dried in a microwave oven, impregnated with Meltilex wax scintillant, and counted at 45% efficiency.

Competition binding assays. Five concentrations of compounds were incubated in triplicate with 18 nM [³H]-histamine and 70 μg of membrane protein under conditions as described above.

Curves were fit to the data with Prism (GraphPad Software) nonlinear least-squares curve-fitting program and Kᵢ values were derived from IC₅₀ values according to Cheng and Prusoff (Cheng, et al., (1973) Biochem. Pharm. 22:3099-3108). The data generated in these experiments is shown, below, in Table 3.

TABLE 3
Compound Potencies Versus [³H]Histamine
Binding to SP9144-Transfected HEK293 Cells.

| Compound | Kᵢ ± SEM¹ (nM) |
|---|---|
| Imetit | 3.1 ± 0.7 |
| Clobenpropit | 7.2 ± 0.5 |
| Histamine | 9.7 ± 0.9 |
| N^{α}-methylhistamine | 63 ± 2 |
| Burimamide | 100 ± 10 |
| (R)-α-methylhistamine | 140 ± 10 |
| Thioperamide | 210 ± 50 |
| Dimaprit | 380 ± 70 |
| (S)-α-methylhistamine | 3400 ± 300 |
| Chlorpheniramine | >10 μM |
| Cimetidine | >10 μM |

¹Standard error of the mean

The methods set forth in this example may be adapted to evaluate the ability of other substances to antagonize histamine H4 receptors.

Methods by which compounds can be evaluated to determine activity at histamine H3 receptors include the guinea pig brain membrane assay and the guinea pig neurogenic ileum contraction assay, both of which are described in U.S. Pat. No. 5,352,707. Another useful assay utilizes rat brain membranes and is described by West, et al., (1990) Molecular Pharmacology 38: 610-613.

A particularly useful screening assay measures binding to sites in guinea pig brain membranes. This test is described in detail by Korte, et al., (1990) Biochem. Biophys. Res. Comm. 168: 979-986, and quantifies the inhibition of radiolabeled N^{α}-methylhistamine binding to tissues by candidate compounds.

Affinity values (Kᵢ) may be determined using the following formula: Kᵢ=IC₅₀/(1+(concentration of ligand/affinity (K_{D}) of radioligand)) The method of Korte, et al (supra) was used to analyze thioperamide and clobenpropit. The results are set forth below in Table 4 (see also WO 98/06394):

TABLE 4
Affinities of THIO and CLOB for the Histamine H3 Receptor.

| Compound | Kᵢ (nM) |
|---|---|
| Thioperamide | 12 |
| Clobenpropit | 0.1 |

### Example 2

### Screening Assay for Histamine H1, H3 and H4 Receptor Antagonists

In the present example, the affinities of several compounds for the H1, H3 and H4 receptors was determined by a membrane binding assay.

Materials. Rat and guinea-pig brains were obtained frozen from Rockland Immunochemicals (Gilbertsville, Pa.). Cell lines expressing recombinant human receptors were generated by using standard transfection techniques. The following radioligands were obtained from Dupont NEN (Boston, Mass.): [³H]-pyrilamine, 23 Ci/mmol, for H1 binding; [³H]-N′-methylhistamine, 82 Ci/mmol, for H3 binding and [³H]-histamine, 20 Ci/mmol, for H4 binding.

Methods. Recombinant cell lines (i.e., human H1-CHO cells, human H3-HEK293 and human H4-HEK293 cells) were cultured in Dulbecco's modified Eagle's medium/10% fetal bovine serum supplemented with 2 mM glutamine, penicillin (100 U/ml), and streptomycin (100 μg/ml) in a humidified 5% CO₂ atmosphere at 37° C. Selection was maintained with 0.5 mg geneticin/ml. Cells were harvested for membrane preparation by aspirating media, replacing it with Hanks' balanced salt solution/5 mM EDTA, and incubating flasks for 10 minutes at 37° C. Cells were pelleted by centrifugation at 1000×g for ten minutes at 4° C.

Membrane preparation. Membranes were prepared by disrupting cells or tissue in at least ten volumes of ice-cold 50 mM Tris-HCl, pH 7.5 at 25° C., with a Polytron. homogenates were centrifuged ten minutes at 1000×g and the supernatants were then centrifuged for ten minutes at 50,000×g. Pellets from this centrifugation step were resuspended with a Polytron, a sample was taken for protein determination (BCA; Pierce; Rockford, Ill.), and the resuspension was again centrifuged at 50,000×g. Brain membranes were stored as pellets, cell membranes as suspensions of 1 mg protein/ml Tris buffer at −20° C.

Binding assays. Membrane (300 μg of brain membrane protein, 5-10 μg of recombinant cell membrane) was incubated with radioligand at a concentration near its K_{D} value without or with inhibitor compounds in a total volume of 200 μl Tris buffer. Nonspecific binding was determined in the presence of 10⁻⁶ M chlorpheniramine for H1 binding, 10⁻⁶ M clobenpropit for H3 binding, or 10⁻⁵ M thioperamide for H4 binding. Assay mixtures were incubated for 30 minutes at 30° C. in polypropylene, 96-well, deep-well plates then filtered through 0.3% polyethylenimine-soaked GF/B filters. These were washed three times with 1.2 ml of Tris buffer, dried in a microwave oven, impregnated with Meltilex wax scintillant and counted at 40% efficiency in a Betaplate scintillation counter (Wallac). IC₅₀ values were determined by interpolation or by nonlinear, least-squares, curve-fitting with the Prism program (GraphPad Software). Kᵢ values were determined in the manner of Cheng and Prusoff (Cheng, et al., (1973) Biochem. Pharm. 22:3099-3108).

The data generated in these experiments are shown, below, in Table 5.

TABLE 5
Equilibrium Dissociation Constants for
the Compounds of Formulas 18-36
at Histamine Receptors H1, H3 and H4.

| Formula | H4 Ki (nM) | H3 Ki (nM) | H1 Ki (nM) |
|---|---|---|---|
| 18 | 34 | 0.06 | |
| 19 | 38 | 55 | 0% |
| 20 | 38 | 2 | |
| 21 | 90 | 3 | |
| 22 | 92 | 0.8 | 330 |
| 23 | 205 | 6 | 660 |
| 24 | 290 | 3 | 29 |
| 25 | 300 | 0.7 | |
| 26 | 320 | 15 | 0% |
| 27 | 325 | 4 | |
| 28 | 390 | 3 | 2 |
| 29 | 440 | 19 | |
| 30 | 480 | 10 | 310 |
| 31 | 770 | 8 | 5 |
| 32 | 850 | 17 | 32% |
| 33 | 870 | 470 | |
| 34 | 1100 | 19 | 48% |
| 35 | 1100 | 7 | 0% |
| 36 | 2% | 1% | 15 |

The methods in the foregoing examples can easily be adapted to determine whether any other substance binds to a histamine H1, H3 or H4 receptor.

### Example 3

### Effect of a Compound Comprising Formula 19 on BAL Cells Recovered from LPS-Challenged Rats

The following example demonstrates the ability of a compound comprising formula 19 (i.e., 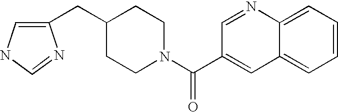
) to reduce the lipopolysaccharide (LPS)-induced inflammatory response in rat airways.

Male Sprague-Dawley rats (250-300 g) were anesthetized by inhalation of isoflurane (flow rate 1 ml/min; supplemented with O₂). Using a Penn-Centry microspray needle, 0.1 ml of a 100-μg/ml LPS solution in saline was injected into the trachea. Animals not challenged with the LPS solution received 0.1 ml of saline. Animals were placed on a heat pad until they recovered from anesthesia. Afterward, they were returned to their cages and allowed food and water ad libitum. All animals survived these manipulations and no additional interventions were required to ensure their survival. Animals fasted overnight were orally dosed with either the standard PD4 inhibitor and positive-control, SB207499 (c4-cyano-4-(3-cyclopentyloxy-4-methoxy-phenyl)-r-1-cyclohexanecarboxylic acid; Barnette, et al., (1998) J. Pharm. Exp. Ther. 284: 420-426), the compound of formula 17 or vehicle as a negative-control (0.4% methylcellulose) five hours before the LPS challenge.

At appropriate time points after intratracheal challenge with LPS, animals were surgically prepared with a tracheal cannula. Surgery was performed under anesthesia. The airways were flushed with 2×2 ml of 0.9% saline and the two washings pooled. Lavage fluid was centrifuged (350 g, 4° C., 7 minutes), supernatant aspirated, erythrocytes lysed, and pellet washed in phosphate-buffered saline containing 10% heat-inactivated fetal calf serum and 10 μg/ml DNase I. The cell suspension was centrifuged, supernatant aspirated, and pellet resuspended in the same buffer. Total cell counts were performed using a Nebauer hemacytometer. Differential cell counts were conducted on Cytospin-prepared slides stained with Fisher's Leukostat stain. At least 200 cells were assessed per slide and standard morphological criteria were used to define neutrophilic cells.

The total number of cells and the number of neutrophils in the broncheoalveolar lavage (BAL) recovered from rats treated with the compound of formula 17, SB207499 or a blank were counted and compared. Fewer cells (i.e., neutrophils or total cells) were counted in the BAL of LPS-challenged rats treated with the compound of formula 17 or with SB207499 than that of rats treated with a blank. The formula 17-dependent and SB207499-dependent inhibition of cellular influx into the BAL indicates that these compounds inhibit the pulmonary inflammation response induced by LPS. The cells identified in the BAL were primarily neutrophils indicating that the inflammatory response induced by LPS was primarily a neutrophilic inflammatory response. The data from these experiments is set forth below in Tables 6 and 7.

TABLE 6
Inhibition of Total Cellular Influx into
BAL in Response to LPS Challenge.

| Treatment | % Inhibition |
|---|---|
| SB207499 (10 mg/kg) | 61.2 |
| Formula 19 (10 mg/kg) | 64.5 |

TABLE 7
Inhibition of Neutrophilic Cellular
Influx into BAL in Response to LPS Challenge.

| Treatment | % Inhibition |
|---|---|
| SB207499 (10 mg/kg) | 68.7 |
| Formula 19 (10 mg/kg) | 71.6 |

The experiments set forth in this example could easily be adapted to test the ability of any other compound or combination of compounds to inhibit pulmonary inflammation.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

1. A method for treating or preventing a condition characterized by airway inflammation in a subject comprising administering one or more histamine H3 receptor antagonists, one or more histamine H4 receptor antagonists and, optionally, one or more histamine H1 receptor antagonists to the subject.

2. The method of claim 1 wherein one or more histamine H3 receptor antagonists are selected from thioperamide, impromidine, burimamide, clobenpropit, impentamine, mifetidine, S-sopromidine, R-sopromidine, clozapine, ciproxifam,

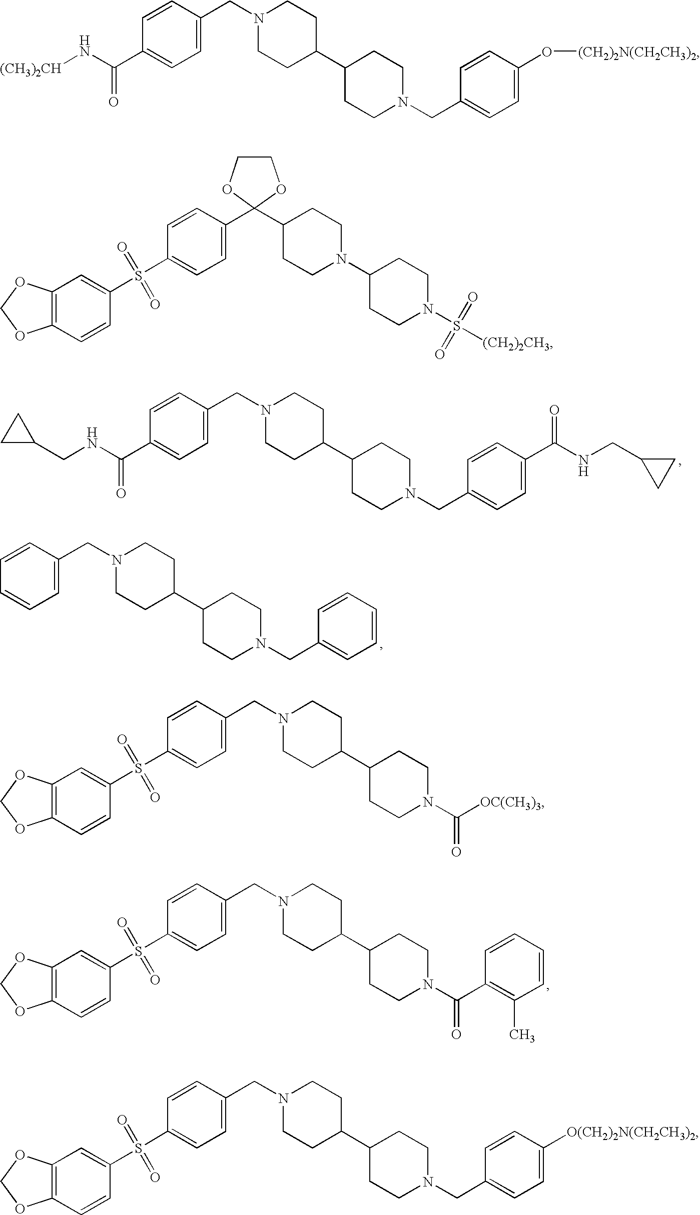

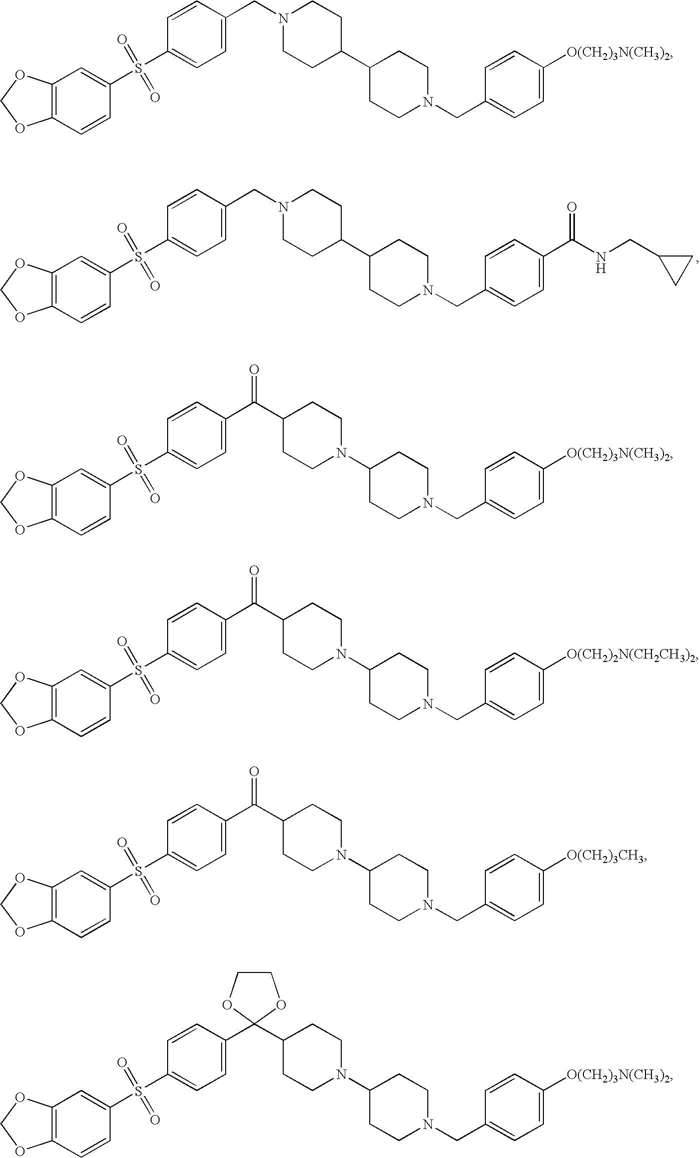

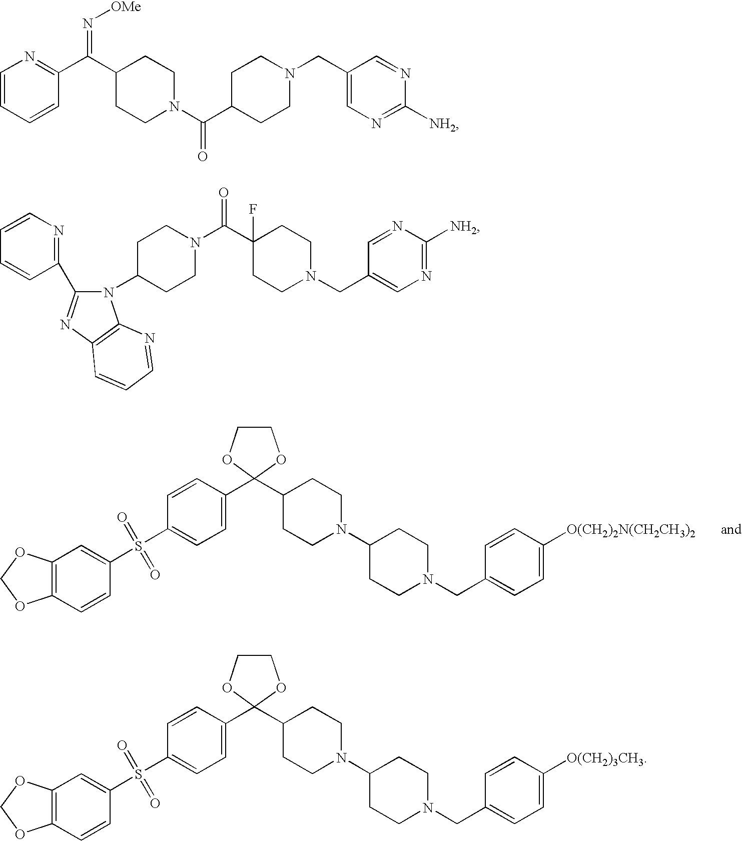

3. The method of claim 1 wherein a histamine H3 receptor antagonist and a histamine H4 antagonist is one or more dual H3/H4 antagonists selected from:

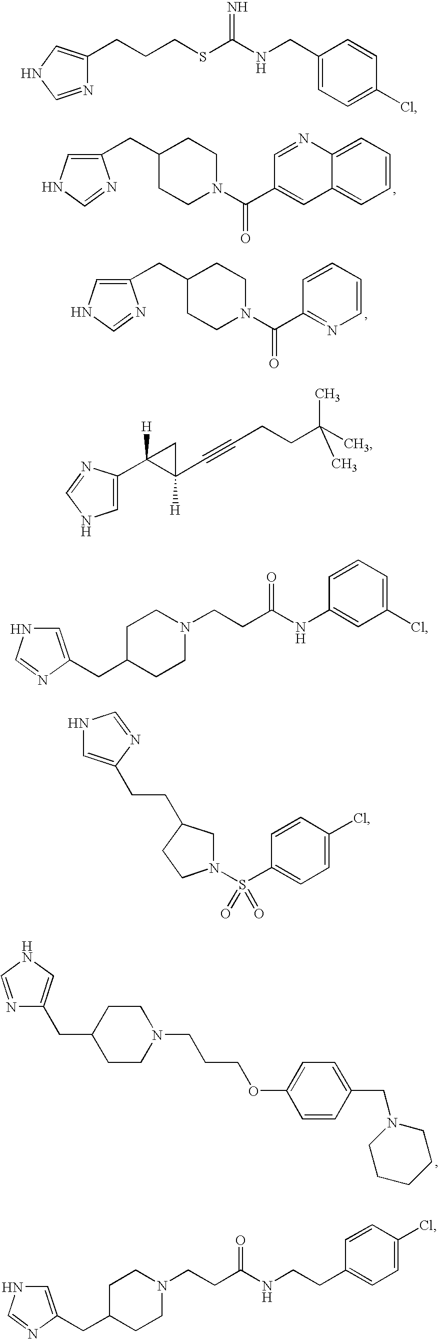

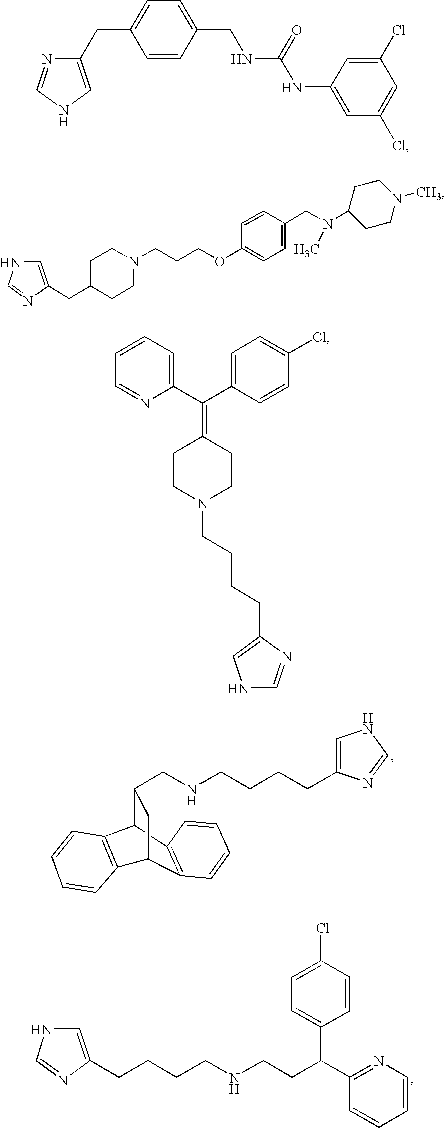

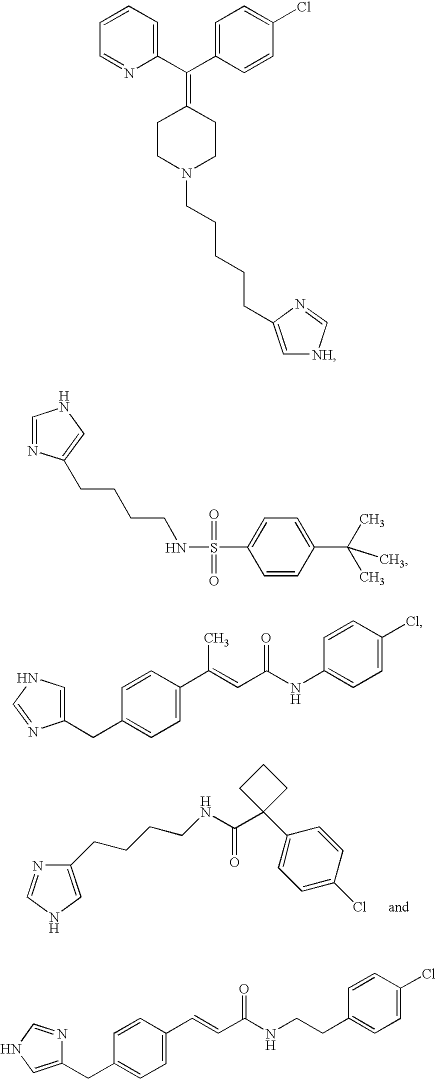

4. The method of claim 1 wherein one or more histamine H1 receptor antagonists are selected from astemizole, azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norasternizole, picumast, pyrilamine, promethazine, terfenadine, tripelennamine, temelastine, trimeprazine, triprolidine and

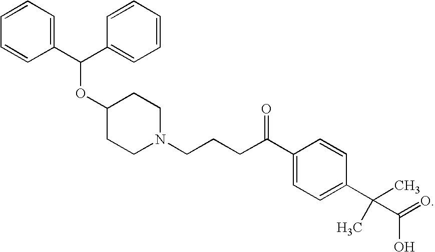

5. The method of claim 4 wherein one or more histamine H1 receptor antagonists are selected from loratadine, desloratadine, cetirizine and fexofenadine.

6. The method of claim 1 wherein one or more of the antagonists are combined with a pharmaceutically acceptable carrier in a pharmaceutical composition.

7. The method of claim 6 wherein the pharmaceutical composition is in the form of a pill, capsule or tablet.

8. The method of claim 1 wherein the subject is a human.

9. The method of claim 1 wherein the condition is selected from allergic rhinitis, congestion and pulmonary inflammation.

10. The method of claim 1 wherein one or more of the antagonists are administered to the subject parenterally.

11. The method of claim 1 wherein one or more of the antagonists are administered to the subject non-parenterally.

12. The method of claim 1 wherein the antagonists are administered in a single composition.

13. A combination comprising
(a) one or more histamine H3 receptor antagonist; in association with
(b) one or more histamine H4 receptor antagonist; and, optionally in association with,
(c) one or more histamine H1 receptor antagonist.

14. The combination of claim 13 wherein one or more histamine H3 receptor antagonists are selected from thioperamide, impromidine, burimamide, clobenpropit, impentamine, mifetidine, S-sopromidine, R-sopromidine, clozapine,

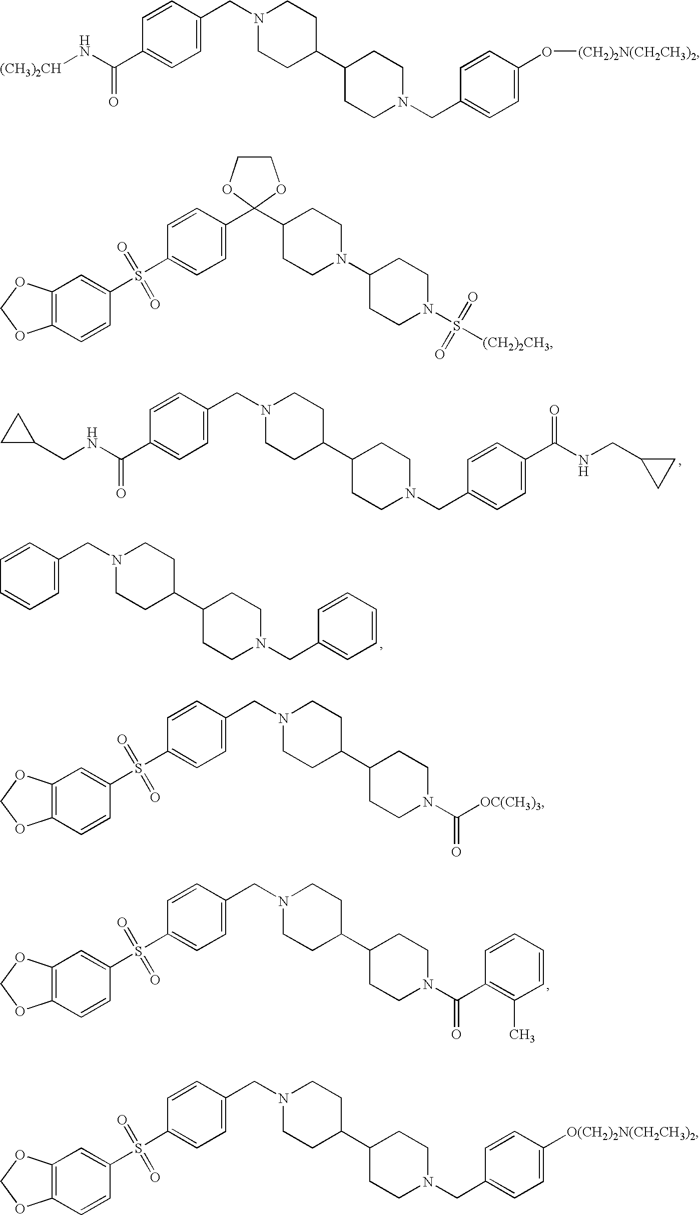

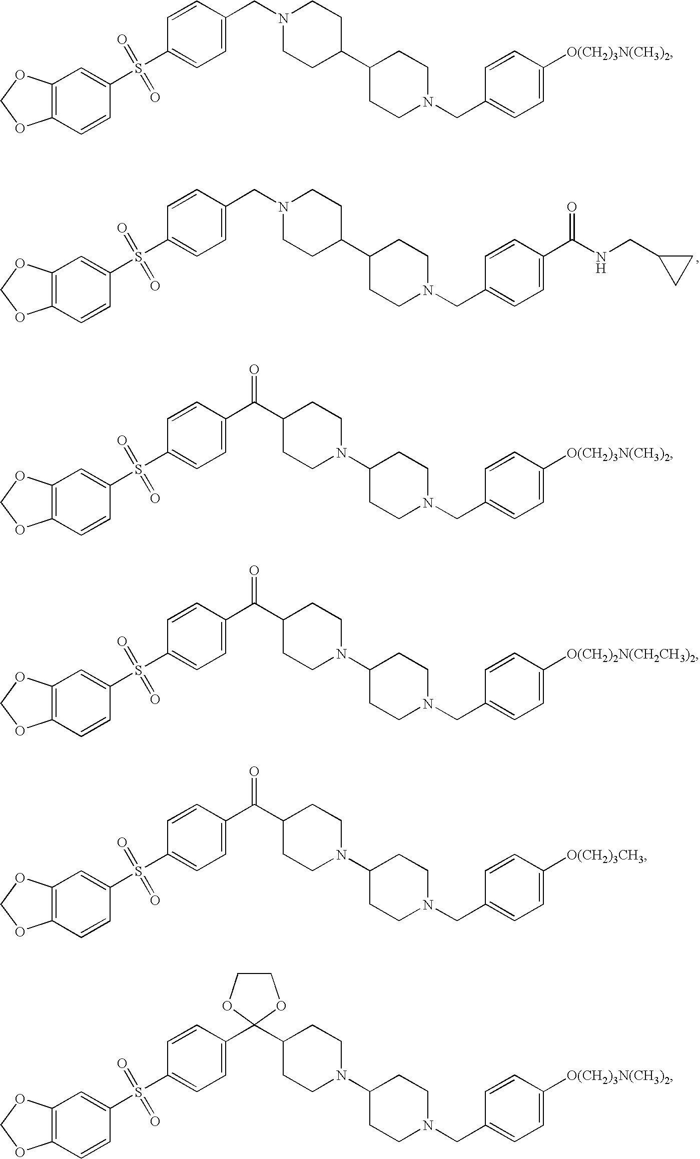

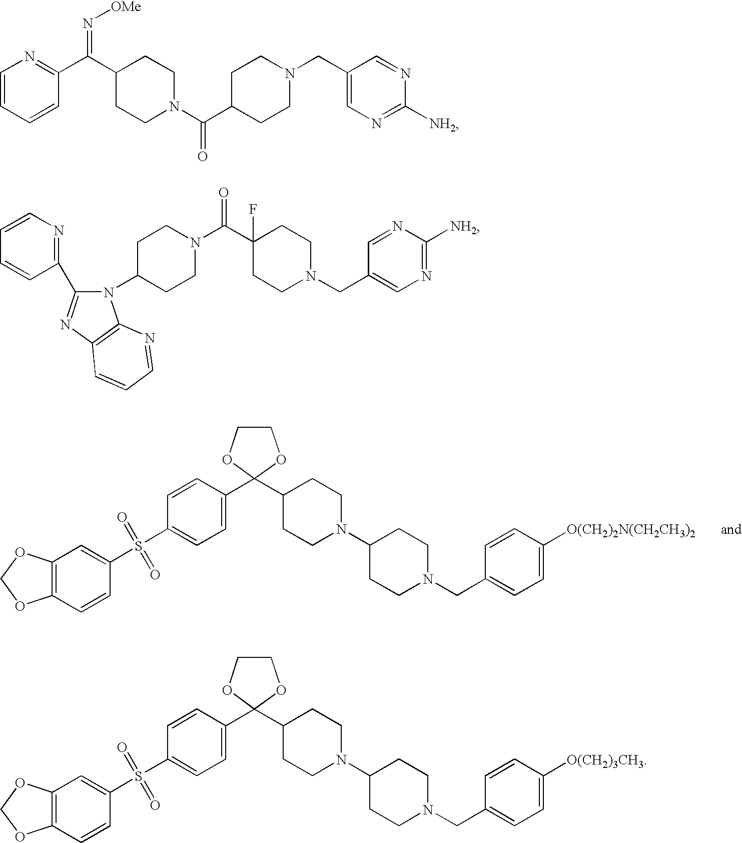

15. The combination of claim 13 wherein the histamine H3 receptor antagonist and the histamine H4 antagonist is one or more dual H3/H4 antagonists selected from

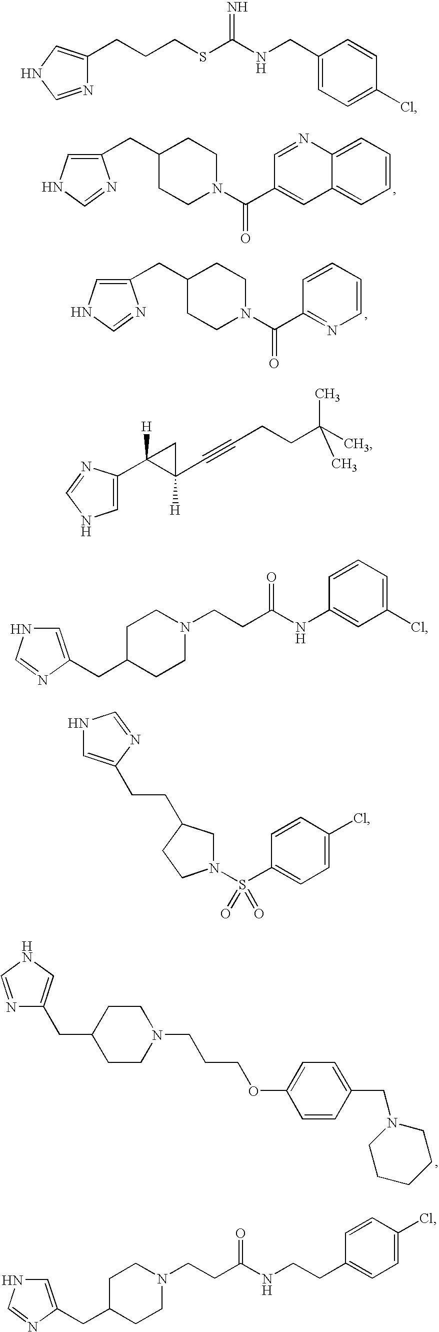

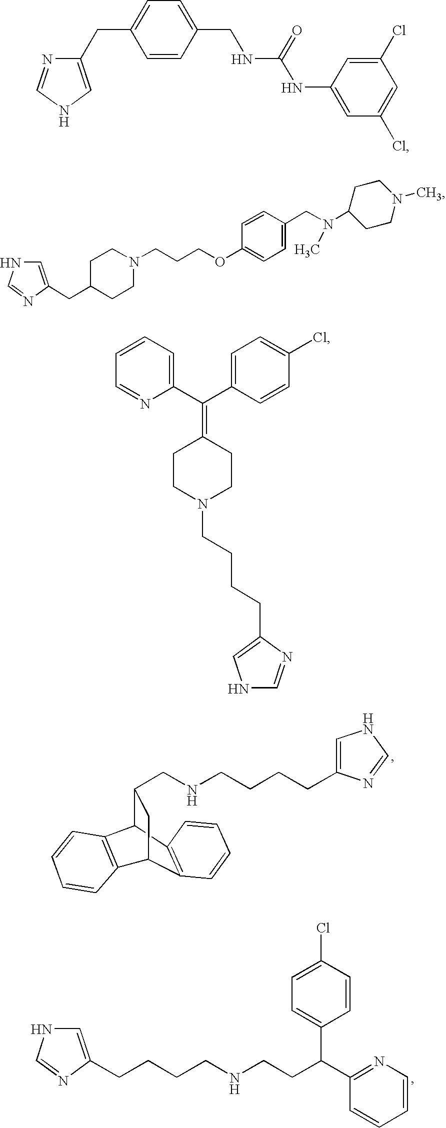

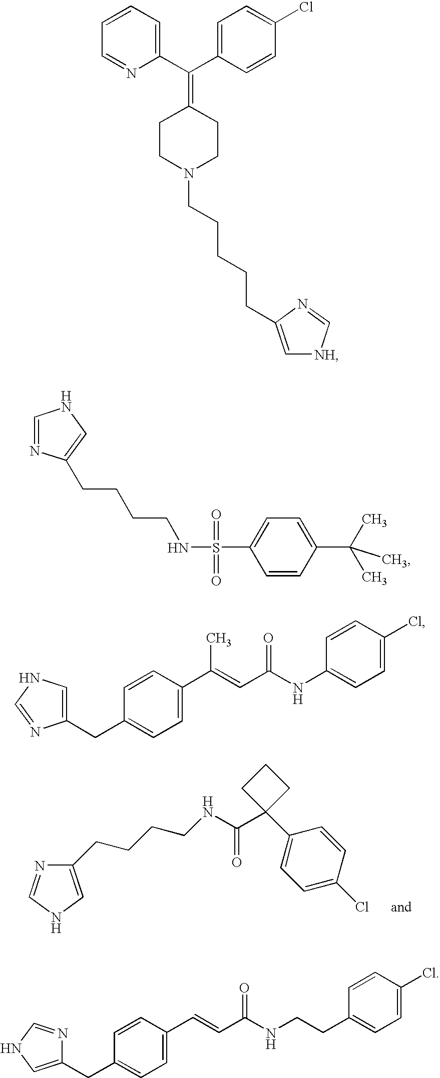

16. The combination of claim 13 wherein one or more histamine H1 receptor antagonists are selected from astemizole, azatadine, azelastine, acrivastine, brompheniramine, cetirizine, chlorpheniramine, clemastine, cyclizine, carebastine, cyproheptadine, carbinoxamine, desloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norasternizole, picumast, pyrilamine, promethazine, terfenadine, tripelennamine, temelastine, trimeprazine, triprolidine and

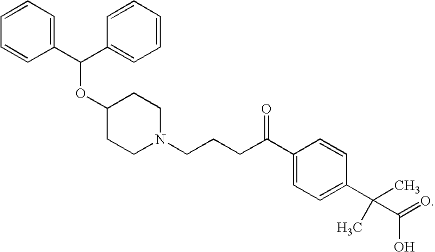

17. The combination of claim 16 wherein one or more histamine H1 receptor antagonists are selected from loratadine, desloratadine, cetirizine and fexofenadine.

18. A pharmaceutical composition comprising a combination of claim 13 and a pharmaceutically acceptable carrier.

19. The composition of claim 18 which is in the form of a pill, capsule or tablet.
